(19) United States
(12) Patent Application Publication
(10) Pub. No.: US 2009/0215102 A1
(43) Pub. Date: Aug. 27, 2009

(54) Method to Assess Breast Cancer Risk

(76) Inventors: Marsha A. Moses, Brookline, MA (US); Susan Pories, Newton, MA (US); David Zurakowski, Boston, MA (US)

Correspondence Address: WOLF GREENFIELD & SACKS, P.C., 600 ATLANTIC AVENUE, BOSTON, MA, 02210-2206, US

(21) Appl. No.: 12/085,134

(22) PCT Filed: Nov. 16, 2006
(86) PCT No.: PCT/US2006/044684

§ 371 Date: Apr. 14, 2009

Related U.S. Application Data
(60) Provisional application No. 60/737,281, filed on Nov. 16, 2005.
(60) Provisional application No. 60/846,456, filed on Sep. 22, 2006.

(51) Int. Cl. C12Q 1/37

(52) U.S. Cl. 435/23

(57) ABSTRACT

We show that urinary metalloproteinases (MMP's) (e.g. MMP 9) and a disintegrin and metalloprotease 12 (ADAM 12) are significantly elevated in women at high risk for developing breast cancer and that monitoring for the absence or presence of both MMP 9 and ADAM 12 represents a new means for breast cancer risk assessment. In addition, we show that levels of MMP 9 and ADAM 12 serve as independent predictors of breast cancer risk. Furthermore, we have determined that elevated levels of urinary ADAM 12 predict an increased risk for breast cancer in subjects predicted not to be at risk for breast cancer by the Gail 5 -year risk model^{66,67}. Accordingly, methods for assessing breast cancer risk and methods for directing medical care are provided.

### CROSS REFERENCE

This Application claims the benefit under 35 U.S.C. §119(e) of U.S. Provisional Application No. 60/737,281 filed Nov. 16, 2005 and U.S. Provisional Application No. 60/846,456 filed Sep. 22, 2006.

### GOVERNMENT SUPPORT

This invention was supported, in part, by National Institutes of Health (NIH) Grants No. PO1CA45548. The government of the United States has certain rights to the invention.

### BACKGROUND OF THE INVENTION

Breast Cancer remains the most common cancer among women and the second leading cause of cancer deaths in women today. The chance of developing invasive breast cancer during a woman's lifetime is approximately one in seven (13.4%). Worldwide, breast cancer is one of the most commonly diagnosed cancers and is the most prevalent cancer in the world¹. The American Cancer Society estimates that, approximately 211,240 women in the United States will be diagnosed with invasive breast cancer and 40,410 women will die of the disease in 2005². Invasive breast cancer alone is responsible for 32% of all new cancer cases in women². Another 58,490 women will be diagnosed with in situ breast cancer, a very early form of the disease . The supposed key to surviving breast cancer is early detection and treatment. Yet breast cancer has been called “an unpredictable disease” because even very small lesions at the limit of detection by mammography or palpation can be shown to have already progressed to metastatic disease³.

Mammography is currently the most sensitive, widely used method of screening women for breast cancer. Though it is currently our “gold standard” for breast cancer detection, mammography is not entirely reliable.³⁹ Certainly, recent advances such as digital mammography have increased diagnostic accuracy and made a substantial difference in reduction in breast cancer mortality.^{40, 41} Yet, in terms of overall diagnostic accuracy, mammography yields a false-negative rate of 10-30% and the sensitivity and accuracy of mammography is compromised in women with high breast density.^{42, 43} False positives are also a substantial problem. In a recent study, it was noted that American mammographers read 10% of all screens as abnormal—and almost all of these are false-positives.^{44, 45} In addition, although most women have access to these tools, they are underutilized by many, including older and economically challenged women, who truly need them.^{46, 47} Recent studies show that breast MRI is superior to mammography for the detection of invasive breast cancer, with twice the sensitivity of mammography and ultrasound.⁴⁸ However, MRIs are expensive, not always covered by health insurance companies and there are considerable differences between institutions with regard to the technique used as well as interpretation of results.

Access to mammography is a serious problem for minority women, low-income women and older women.^{46, 47, 49} Cost, fear of pain and lack of education regarding recommended screening guidelines are also factors that limit the widespread use of mammography.^{50, 51} Mammographic testing requires highly skilled personnel and the purchase and housing of large, expensive pieces of equipment, with attendant maintenance and quality assurance costs. All of these factors may limit accessibility, particularly for disadvantaged women.

Given the limitations of early detection, the goal of identifying women at risk for the disease and providing risk reduction strategies is an especially appealing one. Breast cancer risk assessment is becoming an increasingly significant part of counseling women about their health, especially as successful techniques evolve to predict which women are at the highest risk of developing breast cancer and to prevent the development of a cancerous lesion.

The identification of biomarkers is particularly relevant to improving detection, prognosis, and treatment of breast cancer. As such, there remains a need in the art for alternative biomarkers for the assessment of breast cancer risk that can be quickly, easily, and safely detected. Such biomarkers result in screening methods with high rates of compliance and identify subjects with increased need for subsequent monitoring.

### SUMMARY OF THE INVENTION

We show that urinary metalloproteinases (MMP's) (e.g. MMP 9) and a disintegrin and metalloprotease 12 (ADAM 12) are significantly elevated in women at high risk for developing breast cancer and that monitoring for the absence or presence of both MMP 9 and ADAM 12 represents a new means for breast cancer risk assessment. In addition, we show that levels of MMP 9 and ADAM 12 serve as independent predictors of breast cancer risk. Furthermore, we have determined that elevated levels of urinary ADAM 12 predict an increased risk for breast cancer in subjects predicted not to be at risk for breast cancer by the Gail 5-year risk model^{66,67}. Accordingly, methods for assessing breast cancer risk and methods for directing medical care are provided.

In one aspect, a method for assessing breast cancer risk in a subject is provided where the presence or absence of ADAM 12 and the presence or absence of MMP 9 are detected in a biological sample from a subject. The presence of both ADAM 12 and MMP 9 indicates increased risk of breast cancer. The method may further comprise assessing one or more aspects of the subject's history, such as age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia. In one embodiment, the age of the subject is assessed.

In another aspect, a method for assessing breast cancer risk in a subject deemed to be at low risk for breast cancer according to the Gail 5-year risk model is provided. In one embodiment, the method comprises detecting the level of ADAM 12 in a biological sample from a subject that is at low risk for breast cancer according to the Gail 5-year risk model, and comparing the level to a standard level of ADAM 12, wherein an elevated level of ADAM 12 compared to the standard level indicates increased risk of breast cancer. In one embodiment, the subject that is a low risk for breast cancer according to the Gail 5-year risk model has a score of less than 1.67%.

In another embodiment, the method to assess breast cancer risk in a subject deemed to be at low risk for breast cancer according to the Gail 5-year risk model comprises detecting the level of MMP 9 in a biological sample from a subject that is at low risk for breast cancer according to the Gail 5-year risk model, and comparing the level to a standard level of MMP 9, wherein an elevated level of MMP 9 compared to the standard level indicates increased risk of breast cancer. In one embodiment, the subject that is a low risk for breast cancer according to the Gail 5-year risk model has a score of less than 1.67%.

In one embodiment, the method to assess breast cancer risk in a subject deemed to be at low risk for breast cancer according to the Gail 5-year risk model comprises detecting the level of MMP 9 and the level of ADAM 12 in a biological sample from a subject that is at low risk for breast cancer according to the Gail 5-year risk model, and comparing the level to a standard level of MMP 9 and of ADAM 12, wherein an elevated level of MMP 9 and an elevated level of ADAM 12 compared to the standard level indicates increased risk of breast cancer. In one embodiment, the subject that is a low risk for breast cancer according to the Gail 5-year risk model has a score of less than 1.67%.

In another aspect, a method for assessing breast cancer risk in a patient is provided that comprises measuring the level of ADAM 12 and the level of MMP 9 in multiple biological samples obtained from a subject periodically over a period time. A change in the measured level of ADAM 12 and the measured level of MMP 9 in the biological samples is then measured. An elevation in the measured level of ADAM 12 and/or measured level of MMP 9 over time indicates increased risk of breast cancer. The method may further comprise assessing one or more aspects of the subject's history, such as age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia. In one embodiment, the age of the subject is assessed.

In another embodiment, a method for assessing breast cancer risk in a patient is provided that comprises measuring the level of ADAM 12 in multiple biological samples obtained from a subject periodically over a period time and a change in the measured level of ADAM 12 in the biological samples is then measured. An elevation in the measured level of ADAM 12 over time indicates increased risk of breast cancer. The method may further comprise assessing one or more aspects of the subject's history, such as age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia. In one embodiment, the age of the subject is assessed.

In still another embodiment, a method for assessing breast cancer risk in a patient is provided that comprises measuring the level of MMP 9 in multiple biological samples obtained from a subject periodically over a period time and change in the measured level of MMP 9 in the biological samples is then measured. An elevation in the measured level of MMP 9 over time indicates increased risk of breast cancer. The method may further comprise assessing one or more aspects of the subject's history, such as age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia. In one embodiment, the age of the subject is assessed.

In one embodiment, the biological sample is blood, tissue, serum, urine, stool, sputum, plasma, cerebrospinal fluid, nipple aspirates, or supernatant from cell lysate. In one embodiment, the biological sample is urine.

In other embodiments, the methods for assessing breast cancer risk may further comprise making a decision on the timing and/or frequency of cancer diagnostic testing for the subject, or on the timing and/or prophylactic cancer treatment for the subject.

Methods for directing the medical care of a subject are also provided. In one embodiment, the method to direct medical care comprises assessing risk for breast cancer in a subject using the status of ADAM 12 presence and status of MMP 9 presence in a biological sample from the subject, wherein the presence of both ADAM 12 and MMP 9 indicates increased risk of breast cancer and wherein assessment of increased risk directs medical care comprising a secondary detection method. In one embodiment, the status of MMP 9 presence and the status of ADAM 12 presence are measured by detecting a change in the levels of MMP 9 and a change in the levels of ADAM 12, respectively.

In one embodiment, the method to direct medical care comprises assessing risk for breast cancer in a subject by detecting the level of ADAM 12 in a biological sample from a subject that is at low risk for breast cancer according to the Gail 5-year risk model, and comparing the level to a standard level of ADAM 12, wherein an elevated level of ADAM 12 as compared to the standard level indicates increased risk of breast cancer and wherein assessment of increased risk directs medical care comprising a secondary detection method.

In one embodiment, the method to direct medical care comprises assessing risk for breast cancer in a subject by detecting the level of MMP 9 in a biological sample from a subject that is at low risk for breast cancer according to the Gail 5-year risk model, and comparing the level to a standard level of MMP 9, wherein an elevated level of MMP 9 as compared to the standard level indicates increased risk of breast cancer and wherein assessment of increased risk directs medical care comprising a secondary detection method.

In one embodiment, the method to direct medical care comprises assessing risk for breast cancer in a subject by detecting the level of MMP 9 and the level of ADAM 12 in a biological sample from a subject that is at low risk for breast cancer according to the Gail 5-year risk model, and comparing the level to a standard level of MMP 9 and of ADAM 12, wherein an elevated level of MMP 9 and an elevated level of ADAM 12 as compared to the standard level indicates increased risk of breast cancer and wherein assessment of increased risk directs medical care comprising a secondary detection method.

Secondary detection methods can be, for example, a mammography, an early mammography program, a frequent mammography program, a biopsy procedure, an ultrasound, magnetic resonance imaging, electrical impedance (T-scan) analysis, ductal lavage, ductagram, nuclear medicine analysis, thermal imaging, or any combination of the foregoing.

In one embodiment, the method for directing medical care for a subject comprises assessing risk for breast cancer in a subject using the status of ADAM 12 presence and status of MMP 9 presence in a biological sample from the subject, wherein the presence of both ADAM 12 and MMP 9 indicates increased risk of breast cancer and wherein assessment of increased risk directs medical care comprising breast cancer risk reduction. In one embodiment, the status of MMP 9 presence and the status of ADAM 12 presence are measured by detecting a change in the levels of MMP 9 and a change in the levels of ADAM 12, respectively.

In one embodiment, the method for directing medical care comprises assessing risk for breast cancer in a subject by detecting the level of ADAM 12 in a biological sample from a subject that is at low risk for breast cancer according to the Gail 5-year risk model, and comparing the level to a standard level of ADAM 12, wherein an elevated level of ADAM 12 compared to the standard level indicates increased risk of breast cancer and wherein assessment of increased risk directs medical care comprising breast cancer risk reduction.

In one embodiment, the method for directing medical care comprises assessing risk for breast cancer in a subject by detecting the level of MMP 9 in a biological sample from a subject that is at low risk for breast cancer according to the Gail 5-year risk model, and comparing the level to a standard level of MMP 9, wherein an elevated level of MMP 9 compared to the standard level indicates increased risk of breast cancer and wherein assessment of increased risk directs medical care comprising breast cancer risk reduction.

In one embodiment, the method for directing medical care comprises assessing risk for breast cancer in a subject by detecting the level of ADAM 12 and the level of MMP 9 in a biological sample from a subject that is at low risk for breast cancer according to the Gail 5-year risk model, and comparing the level to a standard level of ADAM 12 and of MMP 9, wherein an elevated level of ADAM 12 and an elevated level of ADAM 12 compared to the standard level indicates increased risk of breast cancer and wherein assessment of increased risk directs medical care comprising breast cancer risk reduction.

Breast cancer risk reduction can be, for example, selective hormone receptor modulator administration, e.g. administration of tamoxifen or raloxifene, or antiangiogenic therapy.

In one embodiment, serial monitoring of the status of ADAM 12 presence and the status of MMP 9 presence is performed at least quarterly, at least bimonthly, at least biweekly, at least weekly, at least every three days, or at least daily.

In one embodiment, serial monitoring of the level of ADAM 12 is performed at least quarterly, at least bimonthly, at least biweekly, at least weekly, at least every three days, or at least daily.

Also provided are methods for monitoring the therapeutic efficacy of a breast cancer risk reduction strategy. In one embodiment, the status of ADAM 12 presence and the status of MMP 9 presence in a biological sample from the subject is used for the assessment of breast cancer risk in the subject, wherein reduction in the levels of ADAM 12 and/or the reduction in the levels of MMP 9 indicates that the breast cancer risk reduction strategy is efficacious. The status of ADAM 12 presence and the status of MMP 9 presence in the biological sample can be measured by detecting a change in the level of ADAM 12 or the level of MMP 9 in the biological sample.

In another embodiment, the method for monitoring the therapeutic efficacy of a breast cancer risk reduction strategy comprises the steps of a) measuring the level of ADAM 12 and the level of MMP 9 in multiple biological samples obtained from a subject periodically over a period time; and b) measuring a change in the measured level of ADAM 12 and a change in the measured level of MMP 9. A reduction in the measured level of ADAM 12 and/or the reduction in the measured level of MMP 9 over time indicates that the breast cancer risk reduction strategy is efficacious.

In one embodiment, the level of ADAM 12 in a biological sample from a subject that is at low risk for breast cancer according to the Gail 5-year risk model is used for the assessment of breast cancer risk in the subject, wherein reduction in the levels of ADAM 12 indicates that the breast cancer risk reduction strategy is efficacious. In another embodiment, the level of MMP 9 in a biological sample from a subject that is at low risk for breast cancer according to the Gail 5-year risk model is used for the assessment of breast cancer risk in the subject, wherein reduction in the levels of MMP 9 indicates that the breast cancer risk reduction strategy is efficacious. In still another embodiment, the level of MMP 9 and the level of ADAM 12 in a biological sample from a subject that is at low risk for breast cancer according to the Gail 5-year risk model is used for the assessment of breast cancer risk in the subject, wherein reduction in the levels of MMP 9 and reduction in the levels of ADAM 12 indicates that the breast cancer risk reduction strategy is efficacious.

Other aspects of the invention include methods to direct treatment of a subject. In one embodiment, the method comprises having a subject tested for the presence of ADAM 12 and the presence of MMP 9 in a biological sample obtained from the subject, wherein a clinician reviews the results and if the biological sample is positive for the presence of ADAM 12 and the presence of MMP 9 the clinician directs the subject to appropriate medical treatment. In one embodiment, multiple biological samples obtained from a subject periodically over a period time are tested and a change in the measured level of ADAM 12 and MMP 9 in the biological samples is measured. If an elevated level of MMP 9 and/or ADAM 12 is detected, the clinician directs the subject to appropriate medical treatment.

In one embodiment, the method to direct treatment of a subject comprises having a subject tested for the level of ADAM 12 in a biological sample a biological sample from a subject that is at low risk for breast cancer according to the Gail 5-year risk model, wherein a clinician reviews the results as compared to a standard level of ADAM 12, and if the biological sample has an elevated level of ADAM 12 as compared to the standard level, the clinician directs the subject to appropriate medical treatment. The appropriate medical treatment can be, for example, medical care comprising a secondary detection method or a breast cancer reduction treatment.

In one embodiment, the method to direct treatment of a subject comprises having a subject tested for the level of MMP 9 in a biological sample a biological sample from a subject that is at low risk for breast cancer according to the Gail 5-year risk model, wherein a clinician reviews the results as compared to a standard level of 19, and if the biological sample has an elevated level of MMP 9 as compared to the standard level, the clinician directs the subject to appropriate medical treatment. The appropriate medical treatment can be, for example, medical care comprising a secondary detection method or a breast cancer reduction treatment.

In one embodiment, the method to direct treatment of a subject comprises having a subject tested for the level of ADAM 12 and the level of MMP 9 in a biological sample a biological sample from a subject that is at low risk for breast cancer according to the Gail 5-year risk model, wherein a clinician reviews the results as compared to a standard level of ADAM 12 and of MMP 9, and if the biological sample has an elevated level of ADAM 12 and an elevated level of MMP 9 as compared to the standard level, the clinician directs the subject to appropriate medical treatment. The appropriate medical treatment can be, for example, medical care comprising a secondary detection method or a breast cancer reduction treatment.

The tests may be performed in the same country where the subject resides or in another country and the results are made available, for example via a Web site, or are transmitted to the clinician.

In one embodiment, the secondary detection method is a mammography, an early mammography program, a frequent mammography program, a biopsy procedure, an ultrasound, magnetic resonance imaging, electrical impedance (T-scan) analysis, ductal lavage, ductagram, nuclear medicine analysis, thermal imaging, or any combination of the foregoing.

In one embodiment, the breast cancer reduction treatment is treatment with a hormone receptor modulator or antiangiogenic therapy.

### BRIEF DESCRIPTION OF THE FIGURES

FIGS. 1A to 1B show a Zymogram and a Western Blot respectively. FIG. 1A, urinary MMPs are present in the urine of women at risk to develop breast cancer: MMP-2 (gelatinase A), MMP-9 (gelatinase B), MMP-9/NGAL complex and the high molecular weight MMP species are the predominant MMP species detectable in human urine by gelatin zymography. FIG. 1B, ADAM 12 is present in the urine of women diagnosed with AH or LCIS and at increased risk of developing breast cancer at significantly higher levels than normal controls. The 68 kDa active form of ADAM 12 is the predominant species detected in human urine by immunoblot analysis using an ADAM 12-specific antibody. Band intensities were analyzed and converted to DU using UN-SCAN-IT™ (Silk Scientific, Orem, Utah) software digitizer technology.⁷³

FIGS. 2A to 2B show theoretical curves illustrating the probability of AH (FIG. 2A) and LCIS (FIG. 2B) as compared to the normal controls on ADAM 12 level. Empirical data are shown as histograms representing the percentage of women in each group with ADAM 12 levels within each of the intervals on the x-axis. Logistic regression analysis indicated a highly significant nonlinear relationship between increasing ADAM 12 level and the increasing probability of AH (LRT=58.4 on 1 degree of freedom, P<0.00011) and LCIS (LRT=53.3 on 1 degree of freedom, P<0.0001). Nearly 60% of controls had ADAM 12 levels of 0, whereas 75% of women diagnosed with AH and 80% with LCIS had ADAM 12 levels greater than 10 densitometric units.

FIG. 3 shows probability curves indicating the combined value of using ADAM 12 level with Gail scores to predict the likelihood of AH. Curves for Gail risk subgroups were derived by multiple logistic regression which confirmed that ADAM 12 level and Gail scores were each independently predictive of an abnormal diagnosis of AH. A woman with a low-risk Gail 5-year score of <1.67% has a low predicted probability of AH if she also has an ADAM 12 level<12 densitometric units.

### DETAILED DESCRIPTION OF THE INVENTION

The present invention is based on the discovery that presence of both ADAM 12 and MMP 9 in the urine of women correlates with a very high incidence of breast lesions that themselves indicate increased risk of developing invasive breast cancer. In a study of 151 women, whereas ADAM 12 and MMP 9 presence individually indicated probabilities of 50-67% and 25-40%, respectively, the presence of both indicated a 100% probability of lesion associated with an increased risk of developing breast cancer. It has also been determined that elevated levels of ADAM 12 indicate an increased risk of breast cancer in subjects that show a low risk of breast cancer according the Gail 5-year risk model.

Accordingly, embodiments of the invention provide methods to assess risk for breast cancer in a subject by detecting, in a biological sample obtained from a subject, the presence or absence of ADAM 12 and the presence or absence of MMP 9. The presence of both MMP 9 and ADAM 12 indicate an increased risk of breast cancer. In one embodiment, the status of MMP 9 presence and the status of ADAM 12 presence are measured by detecting a change in the levels of MMP 9 and a change in the levels of ADAM 12, respectively.

As the level of ADAM 12 and the level of MMP 9 also serve as independent predictors of breast cancer risk, embodiments of the invention further provide methods to assess risk for breast cancer in a subject by measuring the level of MMP 9 or the level of ADAM 12 in a biological sample from a subject, wherein an elevated level of MMP 9, or an elevated level of ADAM 12, indicate an increased risk of breast cancer.

Levels of ADAM 12 and levels of MMP 9 can be measured in multiple biological samples obtained from a subject periodically over a period of time.

Also provided, is a method for assessing breast cancer risk in a subject deemed to be at low risk for breast cancer according to the Gail 5-year risk model. The method comprises detecting the level of ADAM 12 in a biological sample from a subject that is at low risk for breast cancer according to the Gail 5-year risk model, and comparing the level to a standard level of ADAM 12, wherein an elevated level of ADAM 12 compared to the standard level indicates increased risk of breast cancer. In one embodiment, the method comprises detecting the level of MMP 9 in a biological sample from a subject that is at low risk for breast cancer according to the Gail 5-year risk model, and comparing the level to a standard level of MMP 9, wherein an elevated level of MMP 9 compared to the standard level indicates increased risk of breast cancer. In one embodiment, the method comprises detecting the level of MMP 9 and the level of ADAM 12 in a biological sample from a subject that is at low risk for breast cancer according to the Gail 5-year risk model, and comparing the level to a standard level of MMP 9 and of ADAM 12, wherein an elevated level of MMP 9 and an elevated level of ADAM 12 as compared to the standard level indicates increased risk of breast cancer.

Assessment of breast cancer risk provides a means to identify subjects in need of secondary detection methods that can, for example, detect the location of any abnormal lesions or detect the presence of cancerous lesions in the breast.

As used herein, “ADAM 12” or “a disintegrin and metalloproteinase domain 12” or “ADAM metallopeptidase domain 12” refers to the ADAM 12 protein of Genbank accession NM_{—}003474, NM_{—}021641, NP_{—}003465, NP_{—}067673 (Homo sapiens) (SEQ ID NO:1). The term also encompasses species variants, homologues, allelic forms, mutant forms, and equivalents thereof. ADAM 12 is known to mediate cell adhesion and spreading through syndecan and integrin interactions has recently been implicated as a marker for tumor aggressiveness and progression in liver cancer (Pabic et al. Hepatology. 2003; 37(5):1056-1066). ADAM 12 involvement in breast cancer is known (Kveiborg et al. Cancer Res. 2005; 65:4754-61.; Thodeti et al. FEBS Lett. 2005; 579:5589-95.; Roy et al. J Biol. Chem. 2004; 279:51323-30; WO 05/071387) as is its presence in the urine of subjects with non-cancerous lesions (Roy et al. J Biol. Chem. 2004; 279:51323-30). However, the utility of ADAM 12 alone as a biomarker has only been demonstrated for malignant breast cancer.

As used herein, “MMP 9” or “matrix metalloproteinase 9” refers to a 92 kDa Gelatinase (EC 3.4.24.35) of GenBank accession nos. NM-004994, NP-004985. MMP 9 is a secreted protein which was first purified, then cloned and sequenced by Wilhelm et al (1989). A review of MMP 9 by Vu & Werb (1998) provides an excellent source for detailed information and references on this protease. As with other secreted MMPs, MMP 9 is released as an inactive Pro-enzyme or precursor which includes a propeptide domain. The Pro-enzyme is subsequently cleaved to form the active enzyme.

As used herein, the term “protein of interest” refers to ADAM 12 and MMP 9, either separately or together.

As used herein “Atypia” is used interchangeably with “Atypical Hyperplasia” or “AH” or “Atypical Lobular Hyperplasia” or “ALH” or “Atypical Ductal Hyperplasia” or “ADH”. Atypia has been shown to be a major risk factor for future breast cancer development, increasing a woman's relative risk 5.3 times that of the general population. This risk is further increased if the subject has a first-degree relative with breast cancer (10-fold risk).²⁶⁻²⁸ Despite the nomenclature, Lobular Carcinoma In Situ (LCIS) is also considered a marker for increased risk rather than a precursor lesion.^{29, 30} However, it is now being recognized that LCIS may include a spectrum of conditions and that a small subset of these, such as the pleomorphic variant may in fact represent precursor lesions.^{31, 32} The rate of diagnosis of LCIS has increased fourfold in postmenopausal women from 1978 to 1998 as documented in the nine population-based cancer registries that participate in the Surveillance, Epidemiology, and End Results (SEER) Program.²⁹ It appears that the diagnosis of Atypical Hyperplasia (AH) may also be increasing. Rates of atypia are difficult to track as reporting of this diagnosis is not required in tumor registries and this diagnosis is not documented in the SEER data. In the 1980's and 90's, several large studies demonstrated that AH constituted less than 5% of all benign breast biopsies.^{33, 34} In a recent large historical series, atypia represented 4% of all benign breast biopsies.³⁵ More recent reports of atypia rates approach 7% of benign biopsies.³⁶ Tracking of biopsy results at The Mount Auburn Hospital show a doubling in ADH and a three-fold increase in LCIS from 1997-2004 (Table A). This is most likely due at least in part to increased mammographic screening as ADH is identified most frequently with abnormal mammograms.³⁷ It is interesting to note that in a recent prospective study of breast specimens from women at extremely high genetic risk of breast cancer who were undergoing prophylactic mastectomy these high risk lesions were found in 57% of the women.³⁸ Of these, 37% were Atypical Lobular Hyperplasia (ALH), 39% were Atypical Ductal Hyperplasia (ADH), and 25% were LCIS.³⁸ Atypia is diagnosed most often on breast biopsy done for an abnormal mammogram or physical finding but can also be documented with nipple aspiration, random periareolar fine needle aspiration and ductal lavage.⁸

TABLE A
Changing trends in biopsy results

| | | | | | Total |
|---|---|---|---|---|---|
| | ADH/ALH | LCIS | DCIS | Invasive | Biopsies |
| 1997 January-June | 7 (3.3%) | 2 (0.9%) | 16 (7.6%) | 40 (18.9%) | 211 |
| 2004 January-June | 20 (5.7%) | 12 (3.4%) | 20 (5.7%) | 55 (15.6%) | 352 |

As used herein, a “biological sample” refers to a sample of biological material obtained from a subject, preferably a human subject, including a tissue, a tissue sample, a cell sample, e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), and a tumor sample. Biological samples can also be biological fluid samples. In one embodiment the biological sample is urine. However, blood, serum, plasma, saliva, cerebrospinal fluid, nipple aspirates, and supernatant from cell lysate can also be used.

Embodiments of the invention also encompass the use of isolates of a biological sample in the methods of the invention. As used herein, an “isolate” of a biological sample (e.g., an isolate of a tissue or tumor sample) refers to a material or composition (e.g., a biological material or composition) which has been separated, derived, extracted, purified or isolated from the sample and preferably is substantially free of undesirable compositions and/or impurities or contaminants associated with the biological sample.

As used herein, a “tissue sample” refers to a portion, piece, part, segment, or fraction of a tissue which is obtained or removed from an intact tissue of a subject, e.g. a human subject. In one embodiment, the tissue sample is mammary tissue.

A used herein, the term “subject” or “patient” refers generally to a mammal.

In one embodiment, the biological sample is treated as to prevent degradation of protein or mRNA, e.g., ADAM 12 protein, ADAM 12 mRNA, MMP 9 protein, MMP 9 mRNA. Methods for inhibiting or preventing degradation include, but are not limited to, treatment of the biological sample with protease or RNAase inhibitors, freezing the biological sample, or placing the biological sample on ice. Preferably, prior to analysis, the biological samples or isolates are constantly kept under conditions as to prevent degradation of protein or RNA, e.g., ADAM 12 protein, ADAM 12 mRNA, MMP 9 protein, MMP 9 mRNA.

As used herein, the term “increased risk of breast cancer” is used to refer to a risk of breast cancer increased from that of the general population. A person said to have increased risk of breast cancer is in need of monitoring for the development of breast cancer. A person said to have increased risk of breast cancer is in need of monitoring for the continued presence of breast cancer risk markers, for the development of new breast cancer risk markers or the discovery of the presence of breast cancer risk markers. Breast cancer risk markers include the proteins of the present invention, genetic markers of risk, including, but not limited to BRCA1 and BRCA2, family history of breast cancer, smoking habits or past smoking habits, alcohol consumption and other markers of breast cancer risk known to the skilled artisan.

As used herein “serially monitoring” the presence or absence of MMP 9 and ADAM 12, or the levels of ADAM 12 refers to detecting the presence or absence of MMP 9 or ADAM 12, or to measuring the level of ADAM 12 in a sample more than once, e.g., quarterly, bimonthly, monthly, biweekly, weekly, every three days or daily. Serial monitoring includes periodically measuring at regular intervals as deemed necessary by the skilled artisan.

The term “standard level” as used herein refers to a baseline amount of ADAM 12 or MMP 9 as determined from one or more biological samples obtained “normal” or “healthy” subjects believed not to have a cancer. Once a level has become well established for a standard population, results from test biological samples can be directly compared with the known standard level. For example, a baseline may be obtained from at least one subject and preferably is obtained from an average of subjects (e.g., n=2 to 100 or more), wherein the subject or subjects have no prior history of cancer.

As used herein, the term “standard level” is also intended to include a baseline amount of ADAM 12, or MMP 9, as determined in the subject that is to be monitored for breast cancer risk. For example, one need not directly compare the amount of ADAM 12 or MMP 9 in a subject's sample to a standard level derived from normal subjects, rather one can measure a change in levels of ADAM 12 or MMP 9 present in multiple biological samples obtained from the subject over a period of time, e.g. the standard level used for comparison is the level of ADAM 12 or MMP 9 measured in the first biological sample obtained from the subject. An elevation in the measured level of ADAM 12 or MMP 9 over a period of time is indicative of an increased risk of breast cancer. Alternatively, when the therapeutic efficacy of a breast cancer risk reduction strategy is being tested, a reduction in the measured level of ADAM 12 and/or the measured level of MMP 9 over time indicates that the breast cancer risk reduction strategy is efficacious.

As used herein, “a period of time” is intended to include a period of days, weeks, months or even years. Multiple biological samples are obtained from a subject over a period of time, i.e. a biological sample is obtained from a subject periodically over time at various intervals. A biological sample can be obtained from a subject at any interval. For example, a biological sample can be taken every day for weeks, months or years. Alternatively, a biological sample can be obtained once a week, twice a week, three times a week, four times a week, five times a week, or six times a week for a period of weeks, months or years. In one embodiment, a biological sample is obtained once a week over a period of three months. In one embodiment, a biological sample is obtained once a month for a period of months, or years.

For purposes of comparison, the level of ADAM 12 or MMP 9 in a biological sample to be measured is of the same type (obtained from the same biological source) as what is used for determination of the baseline standard level. For example, in one embodiment of the invention, the level of ADAM 12 is measured by measuring the level of ADAM 12 protein in urine. Thus, the baseline standard level is determined by measuring the level ADAM 12 protein in urine from normal, healthy subjects. In another embodiment, the level of ADAM 12 is measured by measuring the amount of ADAM 12 mRNA transcripts in a tissue sample, thus the baseline standard level is determined by measuring the amount of ADAM 12 mRNA transcripts in tissue samples from normal, healthy subjects.

As used herein, “elevation” of a measured level of ADAM 12 or MMP 9 relative to a standard level means that the amount or concentration of ADAM 12 or MMP 9 in a sample is sufficiently greater in a subject's biological sample relative to the standard level of ADAM 12 or MMP 9. For example, elevation of the measured level relative to a standard level may be any statistically significant elevation which is detectable. Such an elevation may include, but is not limited to, about a 1%, about a 10%, about a 20%, about a 40%, about an 80%, about a 2-fold, about a 4-fold, about an 8-fold, about a 20-fold, or about a 100-fold elevation, or more, relative to the standard. The term “about” as used herein, refers to a numerical value plus or minus 10% of the numerical value.

### Detection of ADAM 12 and MMP 9

Detection of ADAM 12 and MMP 9, including measuring levels of expression, as described herein, can be accomplished by any means known to those skilled in the art including, but not limited to, gel electrophoresis, chromatographic techniques, immunoblot analysis, immunohistochemistry, enzyme based immunoassay, mass spectroscopy, high pressure liquid chromatography, surface plasmon resonance, optical biosensors, and/or antibody and protein arrays (Ausubel, F. A. et al., eds., 1990, Current Protocols in Molecular Biology. Greene Publishing and Wiley-Interscience, New York, USA, Chapter 10; Myszka and Rich 2000, Pharm. Sci. Technol. Today 3:310-317).

In one embodiment, antibodies, or antibody equivalents, are used to detect ADAM 12 and MMP 9 proteins in biological samples. In other embodiments, other means for detection of ADAM 12 and MMP 9 expression are used, such as measuring expression by analysis of mRNA transcripts. Measuring mRNA may be preferred, for example when the biological sample is a tumor, or tissue sample. In one embodiment, the, methods used for detecting ADAM 12 are different than the methods used for detecting MMP 9. For example, MMP 9 may be detected by zymography and ADAM 12 may be detected by western blot.

In one embodiment, the methods of the present invention may be performed concurrently with methods of detection for other analytes, e.g., other mRNAs or proteins or small molecules, e.g., other markers associated with cancer risk, e.g., other markers associated with increased breast cancer risk, e.g., other markers associated with cancer, in the biological sample from the subject.

Methods for detecting levels of mRNA are well known to those skilled in the art. For example, detection of RNA transcripts may be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography. Methods to generate probes for hybridization based on the known sequence of the mRNA of interest, e.g., ADAM 12, e.g., MMP 9, are well known to the skilled artisan.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting ADAM 12 mRNA transcripts is described in reference Pabic et. al. Hepatology, 37(5): 1056-1066, 2003, which is herein incorporated by reference in its entirety. Methods to generate primes for amplification based on the known nucleic acid sequence of the gene of interest are well know to the skilled artisan.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Oligonucleotides corresponding to the gene of interest, e.g., ADAM 12 or MMP 9, are immobilized on a chip which is then hybridized with labeled nucleic acids of a test sample obtained from a subject. Positive hybridization signal is obtained with the sample containing transcripts for the gene of interest, e.g., ADAM 12 or MMP 9. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescent-labeled cDNA probes are generated. The microarrays capable of hybridizing to the cDNA of interest, e.g., ADAM 12 or MMP 9, are then probed with the labeled cDNA probes, the slides scanned and fluorescence intensity measured. This intensity correlates with the hybridization intensity and expression levels.

Protein, e.g., ADAM 12 or MMP 9, can also be detected, including measuring protein levels, including measuring protein activity, when the biological sample is a fluid sample such as blood or urine. In one embodiment, protein, e.g., ADAM 12, e.g., MMP 9, is detected by contacting the biological sample with an antibody-based binding moiety that specifically binds to that protein, or to a fragment of that protein. Formation of the antibody-protein complex is then detected and may be measured to indicate protein levels.

The term “antibody-based binding moiety” or “antibody” includes immunoglobulin molecules and immunologically active determinants of immunoglobulin molecules, e.g., molecules that contain an antigen binding site which specifically binds (immunoreacts with) to the protein of interest, e.g., ADAM 12, e.g., MMP 9. The term “antibody-based binding moiety” is intended to include whole antibodies, e.g., of any isotype (IgG, IgA, IgM, IgE, etc), and includes fragments thereof which are also specifically reactive with the protein of interest, e.g., ADAM 12, e.g., MMP 9. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the methods of the invention that use antibody based binding moieties for the detection of the protein of interest, e.g., ADAM 12, e.g., MMP 9, the level of the protein of interest present in the biological samples correlate to the intensity of the signal emitted from the detectably labeled antibody.

In one embodiment, the antibody-based binding moiety is detectably labeled by linking the antibody to an enzyme. The enzyme, in turn, when exposed to it's substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audoradiography. Isotopes which are particularly useful for the purpose of the present invention are ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

As mentioned above, the proteins of the present invention, e.g., ADAM 12, e.g., MMP9, can be detected by immunoassays, such as enzyme linked immunoabsorbant assay (ELISA), radioimmunoassay (RIA), Immunoradiometric assay (IRMA), Western blotting, or immunohistochemistry, each of which are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

### Immunoassays

“Radioimmunoassay” is a technique for detecting and measuring the concentration of an antigen using a labeled, e.g. radioactively labeled, form of the antigen. Examples of radioactive labels for antigens include ³H, ¹⁴C, and ¹²⁵. The concentration of antigen, e.g., ADAM 12, e.g., MMP 9, in a biological sample is measured by having the antigen in the biological sample compete with the labeled, e.g. radioactively, antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” ELISA is a technique for detecting and measuring the concentration of an antigen using a labeled, e.g., enzyme linked, form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody, e.g. anti-ADAM 12, e.g. anti-MMP 9, is linked to a solid phase, i.e. a microtiter plate, and exposed to a biological sample containing antigen, e.g., ADAM 12, e.g., MMP 9. The solid phase is then washed to remove unbound antigen. A labeled antibody, e.g. enzyme linked, is then bound to the bound-antigen, if present, forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen, e.g., ADAM 12, e.g., MMP 9. The antigen-antibody mixture is then contacted with a solid phase, e.g. a microtiter plate, that is coated with antigen, e.g., ADAM 12, e.g., MMP 9. The more antigen present in the sample, the less free antibody that will be available to bind to the solid phase. A labeled, e.g., enzyme linked, secondary antibody is then added to the solid phase to determine the amount of primary antibody bound to the solid phase.

In a “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies, e.g., luciferase, alkaline phosphatase, horseradish peroxidase, or β-galactosidase, or chemical methods, e.g., DAB/Substrate chromagen.

Other techniques may be used to detect the proteins of the present invention, e.g., ADAM 12, e.g., MMP 9, according to a practitioner's preference, based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies, e.g., anti-ADAM 12, e.g., anti-MMP 9, can then be used to detect and/or assess levels of the protein, e.g., ADAM 12, e.g., MMP 9, where the intensity of the signal from the detectable label corresponds to the amount of protein, e.g., ADAM 12, e.g., MMP 9, present. Levels can be quantitated, for example by densitometry.

### Mass Spectometry

In addition, protein, e.g., ADAM 12, e.g., MMP 9, may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to mixed with the known control sample in the same mass spectrometry run.

In one embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined.

In one embodiment, proteins of the invention, e.g., ADAM 12, e.g., MMP 9, are detected by MALDI-TOF mass spectrometry.

### Other Assays

Protein levels, e.g., ADAM 12, e.g., MMP 9, can also be measured by using other biological assays, for example that measure activity, including but not limited to, zymography. Zymography is an assay well known to those skilled in the art and described in Heusen et al., Anal. Biochem., (1980) 102:196-202; Wilson et al., Journal of Urology, (1993) 149:653-658; Hernon et al., J. Biol. Chem. (1986) 261: 2814-2828, Braunhut et al., J. Biol. Chem. (1994) 269: 13472-13479; Moses et al., Cancer Research 58, 1395-1399, Apr. 1, 1998; U.S. patent application Ser. No. 08/639,373, and U.S. Pat. Nos. 6,037,138 and 6,811,995, which are herein incorporated by reference in their entirety.

Methods of detecting ADAM 12 and MMP 9 in a biological sample also include the use of surface plasmon resonance (SPR). In such assays an antibody the binds ADAM 12 or UP 9 need not be detectably labeled and can be used without a second antibody that binds to the specific polypeptide. For example, an antibody specific for ADAM 12 or MMP 9 may be bound to an appropriate solid substrate and then exposed to the sample. Binding of a ADAM 12 or MMP 9 to the antibody on the solid substrate may be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden). Optical biosensors are also contemplated for use in embodiments of the invention.

The SPR biosensing technology has been combined with MALDI-TOF mass spectrometry for the desorption and identification of biomolecules. In a chip-based approach to BIA-MS, a ligand, e.g., a ADAM 12 or MMP 9 antibody, is covalently immobilized on the surface of a chip. Proteins from a sample are routed over the chip, and the relevant are bound by the ligand. After a washing step, the eluted proteins are analyzed by MALDI-TOF mass spectrometry. The system may be a fully automated process and is applicable to detecting and characterizing proteins present in complex biological fluids and cell extracts at low- to subfemtomol levels.

### Antibodies

The antibodies for use in the present invention can be obtained from a commercial source. Anti-MMP antibodies are available from Oncogene Sciences, Cambridge, Mass. Alternatively, antibodies can be raised against the full length polypeptide, or a portion of polypeptide, e.g., ADAM 12, e.g., MMP 9. Methods for the production of ADAM 12 antibodies are disclosed in PCT publication WO 97/40072 or U.S. Application. No. 2002/0182702, which are herein incorporated by reference.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

### Personal History Measures

Additional factors in gauging a subjects risk for developing breast cancer can be used. In particular, one can examine multiple factors including age, ethnicity, reproductive history, menstruation history, use of oral contraceptives, body mass index, alcohol consumption history, smoking history, exercise history, and diet to improve the predictive accuracy of the present methods. A history of cancer in a relative and the age at which the relative was diagnosed with cancer are also important personal history measures. The inclusion of personal history measures with marker data of the presence of MMP 9 and the presence of ADAM12 in an analysis to predict breast cancer is grounded in the understanding that almost all cancers are derived from a dynamic interaction between an individual's genes and the environment in which genes act. In one embodiment, the subject's age is included in the assessment of breast cancer risk.

### Methods to Monitor Breast Cancer Risk

Embodiments of the invention also provide methods for directing medical care for a subject. In one embodiment, the method comprises obtaining a biological sample from the subject and detecting the presence or absence of ADAM 12 and the presence or absence of MMP 9 in the biological sample, detection of the presence of both ADAM 12 and MMP 9 in the biological sample directs medical care comprising a secondary method of detection. In one embodiment, the status of MMP 9 presence and the status of ADAM 12 presence are measured by detecting a change in the levels of MMP 9 and a change in the levels of ADAM 12, respectively.

In one embodiment, the status of ADAM 12 presence and status of MMP 9 presence in a biological sample from the subject are independently used for the assessment of breast cancer risk in the subject.

In another embodiment, the method to direct medical care comprises assessing risk for breast cancer in a subject by detecting the level of ADAM 12 in a biological sample from a subject that is at low risk for breast cancer according to the Gail 5-year risk model, and comparing the level to a standard level of ADAM 12, wherein an elevated level of ADAM 12 as compared to the standard level indicates increased risk of breast cancer and wherein assessment of increased risk directs medical care comprising a secondary detection method. Alternatively, MMP 9 levels or both MMP 9 and ADAM 12 can be measured where elevated levels indicate an increase risk of breast cancer.

In one embodiment, upon evaluation of the risk for breast cancer a secondary detection step is performed.

Secondary detection steps include, but are not limited to, methods to detect or diagnose breast cancer and methods to detect lesions, e.g., atypia, e.g., LCIS, in the subject. Any of a variety of additional detection or diagnostic steps may be used, such as mammography, ultrasound, MRI, electrical impedance (T-scan) analysis of the breast, thermal imaging or any other imaging techniques, biopsy, clinical examination, ductogram, ductal lavage, nuclear medicine analysis, such as scintimammography, thermal imaging of the breast or any other method. In one embodiment, the methods of Int'l App. WO 05/071387 “Methods for diagnosis and prognosis of cancers of epithelial origin” are employed.

In one embodiment, detection regimens are directed for the subject upon assessment of increased risk of breast cancer, for example, a regular mammography regimen, e.g., once a year, or once every six, four, three or two months; an early mammography regimen, e.g., mammography tests are performed beginning at age 25, 30, or 35; one or more biopsy procedures, e.g., a regular biopsy regimen beginning at age 40.

A number of genetic markers have been associated with breast cancer. Examples of these markers include carcinoembryonic antigen (CEA) (Mughal et al., 249 JAMA 1881 (1983)) MUC-1 (Frische and Liu, 22 J. Clin. Ligand 320 (2000)), HER-2/neu (Haris et al., 15 Proc Am Soc Clin Oncology-. A96 (1996)), uPA, PAI-1, LPA, LPC, RAK and BRCA (Esteva and Fritsche, Serum and Tissue Markers for Breast Cancer, in BREAST CANCER, 286-308 (2001)). In one embodiment, genotype analysis for BRCA1, BRCA2, or other breast cancer genetic marker or any combination of breast cancer markers may be employed to further monitor breast cancer risk in a subject.

Additionally, breast cancer risk may be monitored by following ADAM 12 and MMP 9 status in an individual subject. For example, for a subject for whom ADAM 12 status is negative and MMP 9 status is positive, change in the status of ADAM 12 and change in the status of MMP 9 may be monitored in the subject over time.

### Methods to Prevent Breast Cancer

Embodiments of the invention further provide methods for directing medical care for a subject, wherein the subject is directed to medical care for the reduction of breast cancer risk. For example, detection of the presence of both ADAM 12 and MMP 9 in a biological sample from a subject directs medical care to reduce breast cancer risk. In one embodiment, the status of ADAM 12 presence and status of MMP 9 presence in a biological sample from the subject are independently used for the assessment of breast cancer risk in the subject.

In one embodiment, the status of ADAM 12 presence and status of MMP 9 presence in a biological sample from the subject is used for the assessment of breast cancer risk in the subject, wherein upon assessment of increased risk of breast cancer for the subject, medical care for the reduction of risk of breast cancer is directed. In one embodiment, the status of MMP 9 presence and the status of ADAM 12 presence are measured by detecting a change in the levels of MMP 9 and a change in the levels of ADAM 12, respectively.

In one embodiment, the level of ADAM 12 in a biological sample from a subject that is at low risk for breast cancer according to the Gail 5-year risk model is detected and compared the level to a standard level of ADAM 12, wherein an elevated level of ADAM 12 as compared to the standard level indicates increased risk of breast cancer and the assessment of increased risk directs medical care comprising breast cancer risk reduction. Alternatively, MMP 9 levels or both MMP 9 and ADAM 12 can be measured where elevated levels indicate an increase risk of breast cancer.

Breast cancer risk reduction includes preventative prophylactic treatments. For example a preventative prophylactic treatment is prescribed or administered to reduce the probability that a breast cancer associated condition arises or progresses. These treatments sometimes are therapeutic, and sometimes delay, alleviate or halt the progression of breast cancer. Any known preventative or therapeutic treatment for alleviating or preventing the occurrence of breast cancer can be prescribed and/or administered, which include selective hormone receptor modulators, e.g., selective estrogen receptor modulators (SERMs) such as tamoxifen, raloxifene, and toremifene; compositions that prevent production of hormones, e.g., aramotase inhibitors that prevent the production of estrogen in the adrenal gland, such as exemestane, letrozole, anastrozol, groserelin, and megestrol; other hormonal treatments, e.g., goserelin acetate and fulvestrant; biologic response modifiers such as antibodies, e.g., trastuzumab (herceptin/HER2); surgery, e.g., lumpectomy and mastectomy; drugs that delay or halt metastasis, e.g., pamidronate disodium; and alternative/complementary medicine, e.g., acupuncture, acupressure, moxibustion, qi gong, reiki, ayurveda, vitamins, minerals, and herbs, e.g., astragalus root, burdock root, garlic, green tea, and licorice root.

A. Tamoxifen

Tamoxifen (NOLVADEX) a nonsteroidal antiestrogen, is provided as tamoxifen citrate. Tamoxifen citrate tablets are available as 10 mg or 20 mg tablets. Each 10 mg tablet contains 15.2 mg of tamoxifen citrate, which is equivalent to 10 mg of tamoxifen. Inactive ingredients include carboxymethylcellulose calcium, magnesium stearate, mannitol and starch.

Tamoxifen citrate is the trans-isomer of a triphenylethylene derivative. The chemical name is (Z)₂-[4-(1,2-diphenyl-1-butenyl)phenoxy-N,N-dimethylethanamine 2-hydroxy-1,2,3 propanetricarboxylate (1:1). Tamoxifen citrate has a molecular weight of 563.62, the pKa′ is 8.85, the equilibrium solubility in water at 37° C. is 0.5 mg/mL and in 0.02 N HCl at 37° C., it is 0.2 mg/mL.

Tamoxifen citrate has potent antieskogenic properties in animal test systems. While the precise mechanism of action is unknown, the antiestrogenic effects may be related to its ability to compete with estrogen for binding sites in target tissues such as breast. Tamoxifen inhibits the induction of rat mammary carcinoma induced by dimethylbenzanthracene (DMBA) and causes the regression of DMBA-induced tumors in situ in rats. In this model, tamoxifen appears to exert its antitumor effects by binding the estrogen receptors.

Tamoxifen is extensively metabolized after oral administration. Studies in women receiving 20 mg of radiolabeled (TIC) tamoxifen have shown that approximately 65% of the administered dose is excreted from the body over a period of 2 weeks (mostly by fecal route). N-desmethyl tamoxifen is the major metabolite found in patients' plasma. The biological activity of N-desmethyl tamoxifen appears to be similar to that of tamoxifen. 4-hydroxytamoxifen, as well as a side chain primary alcohol derivative of tamoxifen, have been identified as minor metabolites in plasma.

Following a single oral dose of 20 mg, an average peak plasma concentration of 40 ng/mL (range 35 to 45 ng/mL) occurred approximately 5 hours after dosing. The decline in plasma concentrations of tamoxifen is biphasic, with a terminal elimination half-life of about 5 to 7 days. The average peak plasma concentration of N-desmethyl tamoxifen is 15 ng/mL (range 10 to 20 ng/mL). Chronic administration of 10 mg tamoxifen given twice daily for 3 months to patients results in average steady-state plasma concentrations of 120 ng/mL (range 67-183 ng/mL) for tamoxifen and 336 ng/mL (range 148-654 ng/mL) for N-desmethyl tamoxifen. The average steady-state plasma concentrations of tamoxifen and N-desmethyl tamoxifen after administration of 20 mg tamoxifen once daily for 3 months are 122 ng/mL (range 71-183 ng/mL) and 353 ng/mL (range 152-706 ng/mL), respectively. After initiation of therapy, steady state; concentrations for tamoxifen are achieved in about 4 weeks and steady state concentrations for I N-desmethyl tamoxifen are achieved in about 8 weeks, suggesting a half-life of approximately: 14 days for this metabolite.

For patients with breast cancer, the recommended daily dose is 20-40 ma. Dosages greater than 20 mg per day should be given in divided doses (morning and evening). Prophylactic doses may be lower, however.

B. Raloxifene

Raloxifene hydrochloride (EVISTA™) is a selective estrogen receptor modulator (SERM) that belongs to the benzothiophene class of compounds. The chemical designation is methanone, [6-hydroxy-2-(4-hydroxyphenyl)benzo[b]thien-3-yl]-[4-[2-(1-piperidinyl) ethoxy]phenyl]-hydrochloride. Raloxifene hydrochloride (HCl) has the empirical formula C₂₈H₂₇NO₄S.HCl, which corresponds to a molecular weight of 510.05. Raloxifene HCl is an off-white to pale-yellow solid that is very slightly soluble in water.

Raloxifene HCl is supplied in a tablet dosage form for oral administration. Each tablet contains 60 mg of raloxifene HCl, which is the molar equivalent of 55.71 mg of free base.

Inactive ingredients include anhydrous lactose, carnuba wax, crospovidone, ED& C Blue No. 2 aluminum lake, hydroxypropyl methylcellulose, lactose monohydrate, magnesium stearate, I modified pharmaceutical glaze, polyethylene glycol, polysorbate 80, povidone, propylene glycol, and titanium dioxide.

Raloxifene's biological actions, like those of estrogen, are mediated through binding to estrogen receptors. Preclinical data demonstrate that raloxifene is an estrogen antagonist in uterine and breast tissues. Preliminary clinical data (through 30 months) suggest EVISTA lacks estrogen-like effects on uterus and breast tissue.

Raloxifene is absorbed rapidly after oral administration. Approximately 60% of an oral dose is absorbed, but presystemic glucuronide conjugation is extensive. Absolute bioavailability of raloxifene is 2.0%. The time to reach average maximum plasma concentration and bioavailability are functions of systemic interconversion and enterohepatic cycling of raloxifene and its glucuronide metabolites.

Following oral administration of single doses ranging from 30 to 150 mg of raloxifene HCl, the apparent volume of distribution is 2.348 L/kg and is not dose dependent.

Biotransformation and disposition of raloxifene in humans have been determined following oral administration of ¹⁴C-labeled raloxifene. Raloxifene undergoes extensive first-pass metabolism to the glucuronide conjugates: raloxifene-4′-glucuronide, raloxifene-6-glucuronide, and raloxifene-6,4′-diglucuronide. No other metabolites have been detected, providing strong I evidence that raloxifene is not metabolized by cytochrome P450 pathways. Unconjugated raloxifene comprises less than 1% of the total radiolabeled material in plasma. The terminal log linear portions of the plasma concentration curves for raloxifene and the glucuronides are generally parallel. This is consistent with interconversion of raloxifene and the glucuronide metabolites.

Following intravenous administration, raloxifene is cleared at a rate approximating hepatic blood flow. Apparent oral clearance is 44.1 L/kg per hour. Raloxifene and its glucuronide conjugates are interconverted by reversible systemic metabolism and enterohepatic cycling, thereby prolonging its plasma elimination half-life to 27.7 hours after oral dosing.

Results from single oral doses of raloxifene predict multiple-dose pharmacokinetics. Following chronic dosing, clearance ranges from 40 to 60 L/kg per hour. Increasing doses of raloxifene HC 1 (ranging from 30 to 150 ma) result in slightly less than a proportional increase in the area under the plasma time concentration curve (AUC). Raloxifene is primarily excreted in feces, and less than 0.2% is excreted unchanged in urine. Less than 6% of the raloxifene dose is eliminated in urine as glucuronide conjugates.

The recommended dosage is one 60-mg tablet daily, which may be administered any time of day without regard to meals.

Preventative treatments may also include administration of angiogenesis inhibitors. Angiogenesis inhibitors include, but are not limited to, Angiostatin, AVASTIN® (bevacizumab), Arresten, Canstatin, Combretastatin, Endostatin, NM-3, Thrombospondin, Tumstatin, 2-methoxyestradiol, Vitaxin, ZD1839 (Iressa), ZD6474, OS1774 (TARCEVA®/erlotinib), CI1033, PKI1666, IMC225 (ERBITUX®/cetuximab), PTK787, SU6668, SU11248, HERCEPTIN® (trastuzumab), and IFN-α, CELEBREX® (Celecoxib), THALOMID® (Thalidomide), rosiglitazone, bortezomib (VELCADE™), bisphosphonate zolendronate (ZOMETA®), and IFN-α.

Methods for monitoring therapeutic efficacy of a breast cancer risk reduction strategy are also provided. For example, when the status of ADAM 12 presence and the status of MMP 9 presence in a biological sample from the subject is used for the assessment of breast cancer risk in the subject, the reduction in the levels of ADAM 12 and/or the reduction in the levels of MMP 9 indicates that the breast cancer risk reduction strategy is efficacious. Alternatively, a reduction in an elevated level of ADAM 12 in a subject deemed to be at low risk for breast cancer according to the Gail 5-year risk model indicates the breast cancer risk reduction strategy for the subject is efficacious. In one embodiment, a reduction in an elevated level of MMP 9 in a subject deemed to be at low risk for breast cancer according to the Gail 5-year risk model indicates the breast cancer risk reduction strategy for the subject is efficacious. In one embodiment, a reduction in an elevated level of ADAM 12 and a reduction in the level of MMP 9 in a subject deemed to be at low risk for breast cancer according to the Gail 5-year risk model indicates the breast cancer risk reduction strategy for the subject is efficacious.

### Detection Kit

The present invention is also directed to commercial kits for the detection of MMP 9 and ADAM 12. The kit can be in any configuration well known to those of ordinary skill in the art and is useful for performing one or more of the methods described herein for the detection of ADAM 12 and MMP 9. The kits are convenient in that they supply many if not all of the essential reagents for conducting an assay or assays for the detection of ADAM 12 and MMP 9 in a biological sample. In addition, the assay is preferably performed simultaneously with a standard or multiple standards that are included in the kit, such as a predetermined amount of protein or nucleic acid, e.g., ADAM 12, e.g., MMP 9, a standard level, so that the results of the test can be quantitated or validated.

The kits include a means for detecting protein, e.g., ADAM 12, e.g., MMP 9, such as antibodies, or antibody fragments, which selectively bind to that protein, e.g., ADAM 12, e.g., MMP 9, or a set of DNA oligonucleotide primers that allows synthesis of cDNA encoding the protein, or for example, a DNA probe that detects expression of mRNA, e.g., ADAM 12 mRNA, e.g., MMP 9 mRNA. The detection kit is preferentially formulated in a standard two-antibody binding format in which, for example, one ADAM 12-specific antibody captures ADAM 12 in a subject sample and another ADAM-specific antibody is used to detect captured ADAM 12. In another preferred formulation of the detection kit, one MMP 9-specific antibody captures MMP 9 in a subject sample and another MMP 9-specific antibody is used to detect captured MMP 9. For example, the capture antibody is immobilized on a solid phase, e.g., an assay plate, an assay well, a nitrocellulose membrane, a bead, a dipstick, or a component of an elution column. The second antibody, i.e., the detection antibody, is typically tagged with a detectable label such as a calorimetric agent or radioisotope.

In one embodiment, the kit comprises a means for detecting the protein of interest, e.g., ADAM 12, e.g., MMP 9, in a sample of urine. In a specific embodiment, the kit comprises a “dipstick” with antibodies or fragments, e.g., anti-ADAM 12, e.g., anti-MMP 9, immobilized thereon, which specifically bind the protein of interest, e.g., ADAM 12, e.g., MMP 9. Specifically bound protein of interest, e.g., ADAM 12, e.g., MMP 9, can then be detected using, for example, a second antibody that is detectably labeled with a calorimetric agent or radioisotope.

In other embodiments, the detection kits may employ, but are not limited to, the following techniques: competitive and non-competitive assays, radioimmunoassay (RIA), bioluminescence and chemiluminescence assays, fluorometric assays, sandwich assays, immunoradiometric assays, dot blots, enzyme linked assays including ELISA, microtiter plates, surface plasmon resonance, optical biosensors, and immunocytochemistry. For each kit the range, sensitivity, precision, reliability, specificity and reproducibility of the assay are established by means well known to those skilled in the art.

In one embodiment, the detection kit may include means for the detection of other biomarkers, e.g., breast cancer markers, e.g., breast cancer risk markers.

The above described detection kits would further provide instructions for use.

All references cited herein are incorporated herein in their entirety by reference.

### Example I

### Urinary Biomarkers Provide a Novel Non-Invasive Methodology to Assess Breast Cancer Risk

### Methods

### Study Population

Subjects were invited to participate in the study in surgical clinic, radiation oncology clinic and medical oncology clinics at the Beth Israel Deaconess Medical Center, the Mount Auburn Hospital and the Dana Farber Cancer Institute. Normal healthy age matched controls were collected from women who came in for routine screening mammograms at Brigham and Women's Hospital and reported no chronic medical problems, no breast complaints, had a normal mammogram reading and were on no medications. All participants completed a detailed medical history form at the time of urine donation. Pregnant and breast-feeding women were excluded from the study. Risk scores were calculated using the modified Gail model.^{66,67}

### Urine Sample Collection and Processing

Urine collection was performed as previously described.⁴ Samples were collected in sterile containers and immediately frozen at −20° C. Urine was collected according to the institutional bioethical guidelines pertaining to discarded clinical material. Prior to analysis, urine was tested for presence of blood and leukocytes using Multistix 9 Urinalysis Strips (Bayer, Elkhart, Ind.) and samples containing blood or leukocytes were excluded. Creatinine concentration of urine was determined using a commercial kit (Sigma, St. Louis, Mo.) according to the manufacturer's protocol. Protein concentration of urine was determined by the Bradford method using bovine serum albumin as the standard. Urine samples were obtained from 148 women: 44 women with AH, 24 women with LCIS and 80 healthy controls.

### Zymography

Urine samples were processed using a preparation method developed in our laboratory (U.S. patent Ser. No. 08/639,373) and electrophoresed and analyzed using zymograms. Thirty microliters of each urine sample was analyzed by substrate gel electrophoresis (zymography) using gelatin as the substrate as previously reported by us⁴. MMP identity was verified by immunoblot analyses using mono-specific antibodies.

### Western Blot Analysis

Equal amounts of proteins (20 μg) were separated by SDS-PAGE under reducing conditions. Resolved proteins were electrophoretically transferred to nitrocellulose membranes (Schleicher & Schuell, Keene, N.H.) and treated as previously described.⁵ Labeled proteins were visualized with enhanced chemiluminescence (Pierce Chemical Company, Rockford, Ill.). Polyclonal antibodies against human ADAM 12, rb122⁶, S-18 (sc-16526, Santa Cruz Biotechnology, CA) were used at 1 μg/ml and 2 μg/ml concentration respectively. Labeled proteins were visualized with enhanced chemiluminescence (Pierce Chemical Company, Rockford, Ill.). Band intensities were analyzed with UN-SCAN-IT™ (Silk Scientific, Orem, Utah) software digitizer technology. MMP analysis was performed using zymography and ADAM 12 analysis via immunoblotting with monospecific antibodies and subsequent densitometry measurement (DU).

### Mammogram Assessment

The American College of Radiology (ACR) Breast Imaging Reporting and Data System (BI-RADS) was created with the objective of improving communication of mammographic results and providing for outcomes monitoring to improve the quality of subject care.⁷ Assessment categories are as follows. BIRADS 1 indicates a negative mammogram. There is nothing to comment on. The breasts are symmetrical and no masses, architectural disturbances or suspicious calcifications are present. BIRADS 2 indicates benign findings. BIRADS 3 indicates probably benign findings and short interval follow-up recommended. Findings placed in this category should have a very high probability of being benign (≧98%). It is not expected to change over the follow-up interval, but the radiologist would prefer to establish its stability. BIRADS 4 is used to indicate a suspicious abnormality and a biopsy should be considered. More recently this category has been sub-divided into a, b and c indicating an increasing (mild to moderate) level of suspicion. BIRADS 5 is highly suggestive of malignancy and appropriate action should be taken. BIRADS 0 is used when additional imaging evaluation is needed.

Breast Density Categories are described as one of the following: 1. Almost entirely fatty 2. Scattered fibroglandular densities 3. Heterogeneously dense or 4. Extremely dense. Mammograms were evaluated using the standard American College of Radiology (ACR) Breast Imaging Reporting and Data System (BI-RADS).

### Statistical Analysis

Urinary MMPs were compared between AH, LCIS, and normal controls using chi-square analysis. Analysis of variance (ANOVA) with Bonferroni-adjusted comparisons was used to evaluate differences in ADAM 12 levels between the three groups.⁶⁸ Multiple stepwise logistic regression analysis using a backward selection procedure was applied to determine predictors that differentiate AH and LCIS from controls by considering four MMPs, ADAM 12 as a continuous variable, age and Gail scores with the likelihood ratio test (LRT) used to assess statistical significance.⁶⁹ Odds ratios and 95% confidence intervals (CI) for significant predictors were determined using exact methods and probability curves for estimating the likelihood of AH as a function of ADAM 12 levels and Gail 5-year risk scores were derived using regression parameters (slope and intercept coefficients) from the final multivariate model.⁷⁰ Receiver operating characteristic (ROC) curve analysis was applied to assess diagnostic accuracy of ADAM 12, Gail scores and the combination for differentiating AH from normal.⁷¹ Statistical analysis was performed using the SPSS software package (version 14.0, SPSS Inc., Chicago, Ill.). Two-tailed values of P<0.05 were considered statistically significant. Power analysis was conducted a priori and indicated that a minimum sample size of 24 subjects in each of the AH and LCIS groups and 80 controls would provide 90% power (a=0.05, b=0.20) to detect a difference of 20% in the positive expression of each MMP between subjects and controls and using a binomial Z-test for independent proportions⁷² and a 30% difference in mean ADAM 12 levels between the study groups using ANOVA (version 6.0, nQuery Advisor, Statistical Solutions, Saugus, Mass.).

### Results

We have studied urinary expression of MMP-9 and ADAM 12 in 148 women: 44 subjects with ALH/ADH, 24 with LCIS compared to 80 healthy controls.

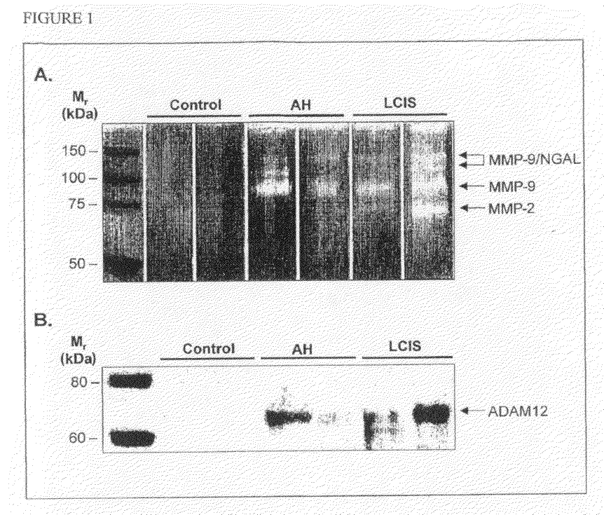

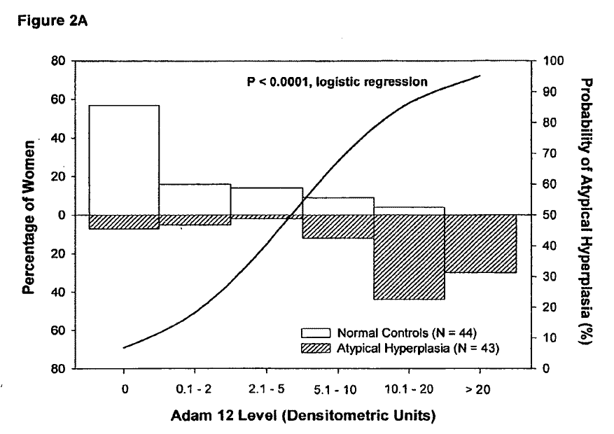

MMP-9, MMP-2, the MMP-9/NGAL complex and ADAM 12 were consistently detected in the urine of the majority of the subjects studied. Representative zymograms for MMP-9 and Western blots for ADAM 12 are shown in FIG. 1.

The majority of the subjects were over age 40 and Caucasian (Table 1). There was no significant difference in smoking or drinking habits between the groups (Table 1). Most of the women studied have never smoked cigarettes or have stopped smoking. Similarly, very few of the women were heavy drinkers.

Mammogram were read as normal with BIRADS scores 1 or 2 in 96% of the normal controls, 63% of the women with LCIS and 38% of the women with atypia. None of the normal controls had mammograms read as BIRADS 4 or 5, which are scores suspicious for or highly suggestive of malignancy while 52% of the women with atypia and 37% of the women with LCIS had mammograms read as BIRADS 4 or 5 (Table 1).

Gail score calculations were consistent with an average risk of breast cancer in the normal controls with a median 5 year risk of 1.0 and were elevated in the subjects with atypia with a median risk of 3.8 (Table 1).

TABLE 1
Characteristics of the Study Groups

| | | Normal | | |
|---|---|---|---|---|
| | | Controls | AH | LCIS |
| | | N = 80 | N = 44 | N = 24 |
| Mean Age - yrs | | 48 ± 8 | 54 ± 8 | 55 ± 9 |
| Age Range - yrs | | 36-76 | 28-77 | 41-85 |
| Race - No. (%) | White | 61 (77) | 42 (95) | 24 (100) |
| | Black | 9 (11) | 0 | 0 |
| | Hispanic | 8 (10) | 0 | 0 |
| | Asian | 2 (2) | 2 (5) | 0 |
| Age at Menarche - yrs | 7-11 | 12 (15) | 7 (16) | 5 (21) |
| | 12-13 | 46 (57) | 26 (59) | 11 (46) |
| | >13 | 20 (25) | 11 (25) | 8 (33) |
| | Unknown | 2 (2) | 0 | 0 |
| Age at 1^{st} Live Birth - yrs | <20 | 9 (11) | 3 (7) | 0 |
| | 20-24 | 14 (17) | 5 (11) | 8 (33) |
| | 25-30 | 14 (17) | 12 (27) | 3 (13) |
| | >30 | 28 (35) | 12 (27) | 5 (21) |
| | Nulliparous | 15 (20) | 12 (27) | 8 (33) |
| Number 1^{st} Degree Relatives | 0 | 62 (78) | 31 (70) | 14 (58) |
| with Breast Cancer | 1 | 11 (14) | 10 (23) | 7 (29) |
| | ≧2 | 1 (1) | 1 (2) | 0 |
| | Unknown | 6 (7) | 2 (5) | 3 (3) |
| Number of Breast Biopsies | 0 | 72 (90) | 12 (27) | 2 (8) |
| | 1 | 8 (10) | 18 (41) | 12 (50) |
| | ≧2 | 0 | 14 (32) | 10 (42) |
| Atypia on Prior Biopsy | No | 8 (10) | 10 (23) | 4 (17) |
| | Yes | 0 | 18 (41) | 16 (67) |
| | Unknown | 0 | 4 (9) | 2 (8) |
| | N/A | 72 (90) | 12 (27) | 2 (8) |
| BIRADS codes | 1 Negative | 69 (86) | 4 (9) | 5 (21) |
| | 2 Benign | 8 (10) | 13 (29) | 10 (42) |
| | 3 probably benign | 3 (4) | 3 (7) | 0 |
| | 4 Suspicious | 0 | 22 (50) | 8 (33) |
| | 5 Highly suggestive of | 0 | 1 (2)* | 0 |
| | N/A | | | |
| Alcohol Intake | None | 12 (15) | 9 (21) | 7 (29) |
| | ≦5 drink/week | 60 (75) | 30 (68) | 15 (63) |
| | >5 drinks/week | 8 (10) | 5 (11) | 2 (8) |
| Smoking | Never smoked | 48 (60) | 29 (66) | 10 (42) |
| | Stopped smoking | 26 (32) | 10 (23) | 10 (42) |
| | <1 pack per day | 4 (5) | 4 (9) | 3 (12) |
| | >1 pack per day | 2 (2) | 1 (2) | 1 (4) |
| Mean Gail Scores‡ - % | 5 Year Risk | 1.0 ± 0.4 | 3.8 ± 1.9† | NA |
| | Lifetime Risk | 10.2 ± 4.1 | 23.2 ± 10.6† | NA |

*This subject did not have a mammogram but MRI was negative
†P < 0.001 for the comparison with normal controls
^{‡}This includes only subjects over the age of 35.

Our results show that MMP-9 and ADAM 12 were independent predictors of both LCIS (both p<0.001) and ADH/ALH (MMP −9, p=0.004; ADAM 12, p<0.001).

### ROC Curve Analysis

Based on intensity units (DU) of ADAM-12, area under the ROC curve was 0.94, which indicates that ADAM-12 is an excellent marker in discriminating breast cancer from normal. Using presence/absence data, the sensitivity is 0.949 (95% CI: 0.827 to 0.994) and the specificity is 0.792 (95% CI: 0.580 to 0.929). The likelihood ratio for a positive test (LR+), determined as sensitivity/[1-specificity], is 4.6, indicating that if a woman tests positive, she is over 4 times more likely to have atypia or LCIS than not.

### Logistic Regression

Logistic regression was performed to determine if a combination of ADAM 12 with MMPs improves diagnostic accuracy and revealed that MMP-9 and ADAM 12 were independent predictors of both LCIS (both p<0.001) and ADH-ALH (MMP-9, p=0.004; ADAM 12, p<0.001).

### Maximum Likelihood Estimation

Maximum likelihood estimation was used to obtain probability of LCIS or ADH-ALH for the various possible test results: a positive urinary MMP-9 and ADAM 12 is associated with a probability of LCIS of 100%, negative for MMP-9 and positive for ADAM 12, a probability of LCIS of 50%, positive for MMP-9 and negative for ADAM 12, a probability of LCIS of 40%, and negative for both MMP-9 and ADAM 12, a probability of LCIS of 0%. Similarly, if a woman's urine is positive for both MMP-9 and ADAM 12, a probability of ADH-ALH is 100%, negative for MMP-9 and positive for ADAM 12, the probability of ADH-ALH is 67%, positive for MMP-9 and negative for ADAM 12, the probability of ADH-ALH is 25%, and a negative results for both MMP-9 and ADAM 12 has an estimated probability of ADH-ALH of 0% (Table 2).

TABLE 2
Maximum Likelihood Estimation

| Test Results | Likelihood of LCIS or Atypia | |
|---|---|---|
| +MMP-9 and +ADAM 12 | 100% | probability of LCIS or atypia |
| −MMP-9 and + ADAM 12- | 50-67% | probability of LCIS or atypia |
| +MMP-9 and −ADAM 12- | 25-40% | probability of LCIS or atypia |
| −MMP-9 and −ADAM 12 | 0% | probability of LCIS or atypia |

### Statistical Analysis

With respect to ADAM 12, univariate analysis indicated that women with AH and LCIS had mean levels of 20.7±16.8 and 14.7±6.9 DU, respectively, which were significantly higher than normal controls (2.1±2.8 DU) as determined by ANOVA with Bonferroni adjustment (both P<0.001). The median ADAM 12 level for normal controls was 0. AH and LCIS groups did not differ significantly from each other in mean or median ADAM 12 level (P>0.20) (Table 3A). There were also significant differences in the percentage of individuals with positive MMP-9 between normal controls and women diagnosed with AH or LCIS (Pearson chi-square=6.17 on 2 degrees of freedom, P<0.05).

Stepwise multiple logistic regression analysis revealed that continuous ADAM 12 level (P<0.0001), positive MMP-9 (P=0.02), and age (P=0.04) were independently predictive in differentiating women diagnosed with AH from controls (Table 3B). The adjusted odds ratio for ADAM 12 in differentiating AH from control is 1.4, implying that each 10-unit increase is associated with an increased odds of 28 times (1.4¹⁰) that the individual has AH rather than being a normal healthy control. This is equivalent to an increased probability of 97%, where probability=odds/(1+odds)=28/29. For binary MMP-9 analysis, an individual who is positive has an estimated risk 5 times higher to have AH than compared to testing negative for MMP-9 (odds ratio=5.1, 95% confidence interval=1.4-17.9). Other variables tested including MMP-2, MMP-9/NGAL, and MMP>150 kDa were not predictive of AH (all P>0.05).

TABLE 3A
Univariate Analysis of Urinary ADAM 12
Levels for Normal Controls and Subject Study Groups

| | | | | Atypical |
|---|---|---|---|---|
| | Normal | Atypical | | Hyperplasia/ |
| | Controls | Hyperplasia | LCIS | LCIS |
| | (N = 80) | (N = 44) | (N = 24) | (N = 68) |
| ADAM 12 | 2.1 ± 2.9 | 20.7 ± 16.8† | 14.7 ± 6.9† | 18.5 ± 14.7† |
| (DU) | 0 (0-3) | 3 (9-27)† | 15 (12-18)† | 13 (11-24)† |
| Median (IQR) | 0-11 | 0-80 | 0-27 | 0-80 |
| Full range | | | | |

Plus-minus values are means ± SD.
DU = densitometric units,
IQR = interquartile range,
ADAM 12, a disintegrin and metalloprotease,
LCIS = lobular carcinoma in situ.
ADAM 12 levels were compared using ANOVA with Bonferroni adjustment for means and the Mann-Whitney U-test for medians.
†P < 0.001 for the comparison with normal controls.

TABLE 3B
Multivariate Logistic-Regression Analysis of Variables Predicting
Atypical Hyperplasia and Lobular Carcinoma In Situ

| | | | |
|---|---|---|---|
| Variable | β Coefficient | Odds Ratio (95% CI) | P Value |

Predictors of Atypical Hyperplasia

| | | | |
|---|---|---|---|
| ADAM 12 | 0.33 | 1.4 (1.2-1.6) | <0.0001 |
| (DU) | | | |
| MMP-9 | 1.62 | 5.1 (1.4-17.9) | 0.02 |
| Age (yr) | 0.08 | 1.1 (1.0-1.2) | 0.04 |

Predictors of Lobular Carcinoma In Situ (LCIS)

| | | | |
|---|---|---|---|
| ADAM 12 | 0.47 | 1.6 (1.3-2.0) | <0.0001 |
| (DU) | | | |
| MMP-9 | 2.61 | 13.8 (1.7-110.7) | 0.01 |
| Age (yr) | 0.09 | 1.1 (1.0-1.2) | 0.05 |

CI = confidence interval.
Other variables including MMP-2, MMP-9/NGAL, and MMP > 150 were not statistically significant (P > 0.05).

When evaluating the predictors of LCIS, logistic regression indicated significant multivariate predictors identical to those for AH, include ADAM 12 (P<0.0001), MMP-9 (P=0.014), and age (P=0.05) (Table 3B). These variables provide independent information in differentiating women with LCIS from normal controls, where the adjusted odds ratio for ADAM 12 is 1.6, implying that each 10-unit increase is associated with an increased odds 110 times (1.6¹⁰) that the individual has LCIS rather than being a normal control. This is equivalent to an increased probability of over 99%. A woman who is positive for MMP-9 has a risk of LCIS over 13 times higher compared to an individual testing negative for MMP-9 (odds ratio=13.8, 95% confidence interval=1.7-110.7). Other variables including MMP-2, MMP-9/NGAL, and MMP>150 kDa were not significant predictors of LCIS (all P>0.05).

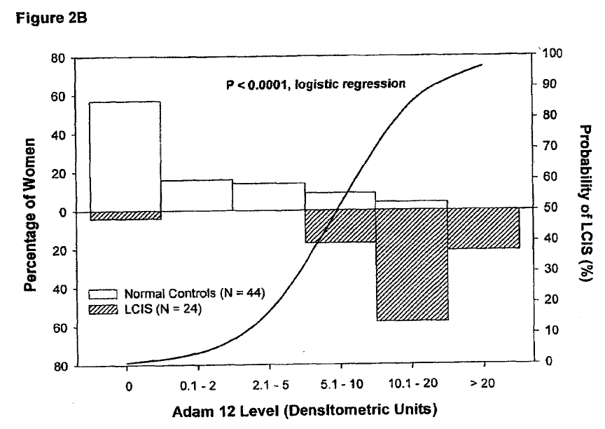

By means of logistic regression analysis, nonlinear equations were derived to estimate the probability of AH and LCIS based on different intervals ADAM 12 and were highly significant for AH (LRT=58.4, P<0.0001) and for LCIS (LRT=53.3, P<0.0001). Empirical data for controls and subjects with a diagnosis of AH (FIG. 2A) or LCIS (FIG. 2B) is represented by bars for each group, reflecting the percentage of women with ADAM 12 levels in each interval. Theoretical curves illustrating the probability of AH or LCIS diagnosis as compared to normal are shown according to ADAM 12 interval in each figure and clearly show the separation between the subjects and controls.

As shown in FIG. 2A, 57% of controls and only 7% of subjects with AH had ADAM 12 levels of 0 in densitometric units, whereas 75% of subjects with AH and only 4% of controls had levels over 10 DU. The predicted probability of AH is 7% for individuals who have ADAM 12 levels of 0, 40% for those with levels between 5-10 DU, and 85% for women with ADAM 12 levels 10-20 DU and 95% for levels over 20 DU. Comparatively, as depicted in FIG. 2B, 57% of controls and only 4% of subjects with LCIS had ADAM 12 levels of 0, whereas almost 80% of subjects with LCIS and only 4% of controls had levels over 10 DU. The predicted probability of LCIS is <5% for individuals with positive ADAM 12 levels<2 DU, 52% for those with levels 5-10 DU, 85% for women with ADAM 12 levels of 10-20 DU, and 97% probability for women with levels over 20 DU.

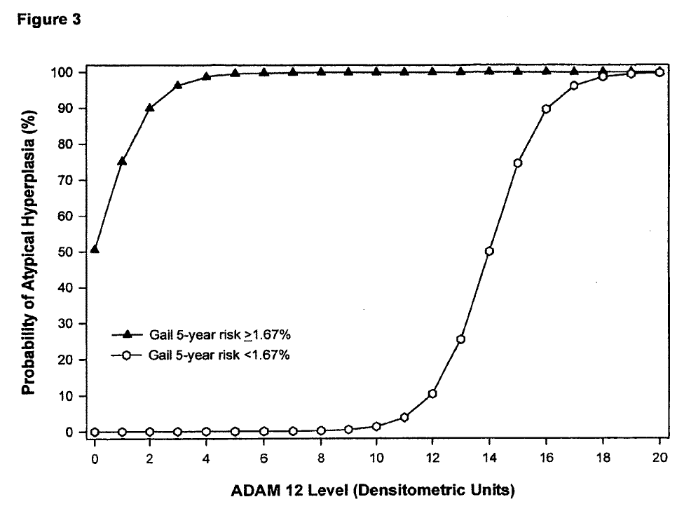

Urinary ADAM 12 levels were then multiplexed with Gail 5-year risk scores, which reflect clinical information.⁶⁷ Gail scores less than 1.67% are considered low risk, while scores equal to or over 1.67% are high risk for the development of breast cancer. Multiple logistic regression analysis identified ADAM 12 level (LRT=19.92, P<0.0001) as a significant predictor for differentiating AH from controls. The results of this modeling approach can be seen as the increasing probability of AH with increasing ADAM 12 levels separately according to high or low Gail-5-year risk (FIG. 3). For example, the probability of AH for an ADAM 12 level of 2 DU is 90% for women who have Gail scores≧1.67% and essentially 0% for those with low-risk Gail scores<1.67%. On the other hand, the probability of AH in women with low-risk Gail scores<1.67% (bottom curve) starts to increase with moderately high ADAM 12 levels (e.g., levels of 12 DU or higher). For example, ADAM 12 levels of 14 and 15 DU are associated with probabilities of 50% and 75%, respectively in this subgroup of women with low-risk Gail 5-year scores. There is an estimated 90% or higher probability of AH in individuals with Gail 5-year risk <1.67% and ADAM 12 levels of 16 DU or greater.

Similarly, ROC analysis of continuous ADAM 12 levels alone, demonstrates excellent discrimination in differentiating women with AH from normal controls, with area under the ROC curve of 0.914. Urinary ADAM 12 levels also provide exceptional discrimination in differentiating women with LCIS from normal controls, with area under the curve (AUC) of 0.950. As the maximum AUC is 1.0 for any test, these scores demonstrate the enormous predictive power of urinary ADAM 12 levels.

When urinary ADAM 12 levels are multiplexed with the clinical information that Gail 5-year risk scores provide, further ROC analysis indicates that the optimal combination is Gail+0.15×ADAM 12 (AUC=0.996). Therefore, the best performance is obtained when both ADAM 12 and the Gail risk score are used together. The best cutoff for this combination index is 2.8. Using the cutoff of 2.8, the sensitivity for the combination is 0.976 (41 out of 42 AH cases were classified correctly) and specificity is 0.977 (43 of 44 controls were classified correctly), Consequently, use of this ADAM 12-Gail combination index in our population yields only one false positive (one control has a combination index of 2.95, scoring above 2.8) and one false negative (one woman with AH has a combination of 2.3, scoring below the 2.8 cutoff).

The development of alternative and improved methods to identify women at high risk for breast cancer is an area of intense investigations. We have chosen to focus on MMPs as biomarkers as they play a role in the earliest stages of breast cancer progression. In this study, we look at the expression of urinary MMPs as well as urinary ADAM 12 in subjects with biopsy proven atypical hyperplasia and lobular carcinoma in situ. Both atypia and lobular carcinoma in situ (LCIS) are recognized as pathologic indicators of increased risk. As the options for breast cancer risk reduction improve, the precise identification of women at increased risk for developing breast cancer becomes increasingly important. Our results indicate that urinary biomarkers ADAM 12 and MMP-9 are highly significant predictors of AH and LCIS.

MMPs are matrix-degrading enzymes that play an important role in tumor growth, metastasis, and the remodeling of the tumor microenvironment. Immunohistochemical studies demonstrate elevated levels of MMP expression in breast tumors⁹⁻¹¹ and breast tumor extracts have been shown to contain active MMPs⁴. Elevated levels of MMP-9 have been reported in plasma of subjects with breast cancer¹². We first observed that functional urinary MMPs could be detected by zymography (substrate gel electrophoresis) in urine of mice implanted subcutaneously with Lewis lung carcinoma tumor cells. Zymography displays enzymatic activities by electrophoretic separation, and allows individual matrix-degrading components to be visualized on gels. Importantly, these original data suggested that MMPs, despite their size, were filtered through the urinary collecting system in tumor-bearing hosts and could be stored in a bioactive form in the bladder. This surprising result indicated that MMPs were likely to be present in urine from individuals bearing tumors at sites distant from the urinary bladder. Overproduction of MMPs by a tumor in communication with the vascular and lymphatic systems might result in increased levels of MMP activity in other body fluids, such as blood or urine. This possibility is consistent with previous demonstrations that levels of other regulatory molecules overproduced by tumors, such as the angiogenic peptide bFGF, have been measured in body fluids of cancer subjects and have been shown to be independent predictors of disease status.^{13, 14} We subsequently tested this hypothesis further in human subjects.

We went on to report that intact MMPs can be detected in the urine of breast cancer subjects and that these urinary MMPs are predictive of disease status⁴. Since our original report, there are now many other independent published studies that support our findings that urinary MMPs predict neoplastic disease status.^{5, 15}-22

Subsequently, we demonstrated that MMPs are required for the switch to the angiogenic phenotype, an early and critical event in cancer growth and progression.²³ Furthermore, we have recently identified a novel high molecular weight urinary MMP as being a complex of MMP-9/NGAL (Neutrophil Gelatinase Associated Lipocalin).⁵ We have examined the urinary MMP expression in a large series of subjects and demonstrated that there is an significant increase in the appearance of MMPs in the urine of subjects as the disease progresses. We have also observed that the MMP species that appear most frequently in the urine of breast cancer subjects is the 92 kDa species (MMP-9). Interestingly, analysis of the identity of MMPs detected in the urine of these breast cancer subjects revealed a significant lack of MMP-2 (72 kDa gelatinase), in contrast to results in a separate study of bladder and prostate cancer subjects in which we detected a high frequency of MMP-2. This suggests that there may be a specific tumor “fingerprint” based on the appearance of different MMP species in the urine of these subjects.

Given these findings and our interest in urinary disease markers we have established a biomarker identification initiative to identify proteins present in urine of cancer subjects and to determine whether their presence might be relevant to disease status. Most recently, we have isolated and identified ADAM 12 (a disintegrin and metalloproteinase) in urine from breast cancer subjects.²⁴ ADAM 12 is part of a glycoprotein family that are related to MMPs. Increased expression of ADAM 12 has previously been reported in breast, colon, and lung carcinoma tissues.25 We established the substrate specificity of ADAM 12 and showed that this enzyme can degrade gelatin, Type IV collagen and fibronectin but not Type I collagen or casein, which suggests that this enzyme plays a role in ECM remodeling, a hallmark of neoplastic disease. We have also demonstrated that urinary ADAM 12 also significantly increases with disease progression in breast cancer subjects and correlates with stage of disease. ADAM 12 was undetectable or present at very low levels in normal controls and increased in subjects with atypia and LCIS as well as invasive cancer. The highest levels of ADAM 12 were found in metastatic disease.²⁴

Here, we report the multiplexing of these two powerful urinary indicators of breast cancer disease progression: MMP-9 and ADAM-12. When urinary MMP-9 and ADAM 12 results are multiplexed and both are positive, a highly significant correlation with proven risk factors for the development of breast cancer; atypia and LCIS is seen.

Atypia has been shown to be a major risk factor for future breast cancer development, increasing a woman's relative risk 5.3 times that of the general population. This risk is further increased if the subject has a first-degree relative with breast cancer (10-fold risk).²⁶⁻²⁸ Despite the nomenclature, LCIS is also considered a marker for increased risk rather than a precursor lesion.^{29, 30} However, it is now being recognized that LCIS may include a spectrum of conditions and that a small subset of these, such as the pleomorphic variant may in fact represent precursor lesions.^{31, 32} The rate of diagnosis of LCIS has increased fourfold in postmenopausal women from 1978 to 1998 as documented in the nine population-based cancer registries that participate in the Surveillance, Epidemiology, and End Results (SEER) Program.²⁹ It appears that the diagnosis of Atypical Hyperplasia (AH) may also be increasing. Rates of atypia are difficult to track as reporting of this diagnosis is not required in tumor registries and this diagnosis is not documented in the SEER data. In the 1980's and 90's, several large studies demonstrated that AH constituted less than 5% of all benign breast biopsies.^{33, 34} In a recent large historical series, atypia represented 4% of all benign breast biopsies.³⁵ More recent reports of atypia rates approach 7% of benign biopsies.³⁶ Tracking of biopsy results at The Mount Auburn Hospital show a doubling in ADH and a three-fold increase in LCIS from 1997-2004 (Table 8). This is most likely due at least in part to increased mammographic screening as ADH is identified most frequently with abnormal mammograms.³⁷ It is interesting to note that in a recent prospective study of breast specimens from women at extremely high genetic risk of breast cancer who were undergoing prophylactic mastectomy these high risk lesions were found in 57% of the women.³⁸ Of these, 37% were Atypical Lobular Hyperplasia (ALH), 39% were Atypical Ductal Hyperplasia (ADH), and 25% were LCIS.³⁸

TABLE 8
Changing trends in biopsy results

| | | | | | Total |
|---|---|---|---|---|---|
| | ADH/ALH | LCIS | DCIS | Invasive | Biopsies |
| 1997 January-June | 7 (3.3%) | 2 (0.9%) | 16 (7.6%) | 40 (18.9%) | 211 |
| 2004 January-June | 20 (5.7%) | 12 (3.4%) | 20 (5.7%) | 55 (15.6%) | 352 |

Atypia is diagnosed most often on breast biopsy done for an abnormal mammogram or physical finding but can also be documented with nipple aspiration, random periareolar fine needle aspiration and ductal lavage.⁸ More reliable, less invasive and less expensive approaches for assessing the risk of breast cancer are needed. Mammography is currently the most sensitive, widely used method of screening women for breast cancer. Though it is currently our “gold standard” for breast cancer detection, mammography is not entirely reliable.³⁹ Certainly, recent advances such as digital mammography have increased diagnostic accuracy and made a substantial difference in reduction in breast cancer mortality.^{40, 41} Yet, in terms of overall diagnostic accuracy, mammography yields a false-negative rate of 10-30% and the sensitivity and accuracy of mammography is compromised in women with high breast density.^{42.43} False positives are also a substantial problem. In a recent study, it was noted that American mammographers read 10% of all screens as abnormal—and almost all of these are false-positives.^{44, 45} In addition, although most women have access to these tools, they are underutilized by many, including older and economically challenged women, who truly need them.^{46, 47} Recent studies show that breast MRI is superior to mammography for the detection of invasive breast cancer, with twice the sensitivity of mammography and ultrasound.⁴⁸ However, MRIs are expensive, not always covered by health insurance companies and there are considerable differences between institutions with regard to the technique used as well as interpretation of results.

Access to mammography is a serious problem for minority women, low-income women and older women.^{46, 47, 49} Cost, fear of pain and lack of education regarding recommended screening guidelines are also factors that limit the widespread use of mammography.^{50, 51} Mammographic testing requires highly skilled personnel and the purchase and housing of large, expensive pieces of equipment, with attendant maintenance and quality assurance costs. All of these factors may limit accessibility, particularly for disadvantaged women.

Presently, Tamoxifen is the only FDA approved agent for breast cancer risk reduction. For women with a history of LCIS the risk is reduced 56% and with a history of atypical ductal hyperplasia, Tamoxifen reduces the risk even more significantly—by 86%.⁵² Although generally well tolerated, Tamoxifen does have associated toxicity, including an increased risk of endometrial cancer, stroke, pulmonary emboli, and deep vein thrombosis, particularly for women aged 50 or older.⁵² Consequently other trials, most notably the STAR trial, (the Study of Tamoxifen and Raloxifene) are ongoing to identify better medical options for high risk women.^{53, 54} The STAR trial, (the Study of Tamoxifen and Raloxifene) is a direct comparison of the two drugs in a trial designed to assess the reduction in the risk of invasive breast cancer.^{55, 56} Raloxifene, a second generation selective estrogen modulator (SERM) a drug currently labeled for the prevention of osteoporosis has also been demonstrated to have anti-estrogenic properties, with minimal stimulation of endometrial epithelium. This 5-year study conducted by the NSABP, was designed to enroll 19,000 postmenopausal women with a Gail relative risk of at least 1.67%. The STAR trial will determine which therapy most significantly reduces invasive breast cancer and will establish a risk benefit profile for each drug with endpoints including endometrial cancer, heart disease, bone fractures and quality of life. Accrual to the STAR trial has closed and results should be available in 2006. In addition promising new agents, such as estrogen synthetase inhibitors are now being evaluated as possible breast cancer risk reduction agents.⁵⁷⁻⁶⁰

Currently, there are insufficient clinical markers available for reliable detection of the presence of a primary breast lesion (screening), for monitoring clinical course, and for determining which subsets of subjects could benefit from more aggressive therapies. While mathematical models such as the Gail model⁶¹, Claus model⁶² and the BRACAPRO model⁶³ are useful, a dependable biomarker would clearly be the most desirable method for identifying risk and tracking progression of breast cancer. Recently, there has been increasing interest in MMPs as biomarkers for cancer diagnosis and monitoring.^{64, 65} The present method for assessing breast cancer risk with a non-invasive urine test is enormously appealing. Evaluating urine proteins is less invasive, awkward, and costly than a mammogram, it should be more easily tolerated by most women, and it could encourage higher compliance with screening efforts. This could be used to start risk reduction efforts before mammographic changes or breast masses even appear.

Our data clearly shows that urinary ADAM 12 and MMP-9 are highly significant predictors of breast cancer risk markers AH and LCIS. ADAM 12 levels were found to provide excellent discrimination in differentiating women with AH or LCIS from normal controls. When ADAM 12 levels are multiplexed with the Gail risk score, the resultant index is even more accurate in distinguishing between normal controls and AH for women categorized as low risk by the Gail model alone (sensitivity 0.976, specificity 0.977). This urinary biomarker approach can be used to identify subjects who would benefit from early risk reduction efforts before mammographic changes or breast masses even appear.

The references cited below and throughout the specification are incorporated herein in their entirety.

### REFERENCES

1. Parkin D M, Bray F, Ferlay J, Pisani P. Global cancer statistics, 2002. CA Cancer J Clin 2005; 55:74-108.
2. Jemal A, Murray T, Ward E, et al. Cancer statistics, 2005. CA Cancer J Clin 2005; 55:10-30.
3. Strategies for Managing the Breast Cancer Research Program: A Report to the U.S. Army Medical Research and Development Command.: Institute of Medicine National Academy Press, 1993.
4. Moses M A, Wiederschain D, Loughlin K R, Zurakowski D, Lamb C C, Freeman M R. Increased incidence of matrix metalloproteinases in urine of cancer subjects. Cancer Res 1998; 58:1395-9.
5. Yan L, Borregaard N, Kjeldsen L, Moses M A. The high molecular weight urinary matrix metalloproteinase (MMP) activity is a complex of gelatinase B MMP-9 and neutrophil gelatinase-associated lipocalin (NGAL). Modulation of MMP-9 activity by NGAL. J Biol Chem 2001; 276:37258-65.
6. Gilpin B J, Loechel F, Mattei M G, Engvall E, Albrechtsen R, Wewer U M. A novel, secreted form of human ADAM 12 (meltrin alpha) provokes myogenesis in vivo. J Biol Chem 1998; 273:157-66.
7. Lacquement M A, Mitchell D, Hollingsworth A B. Positive predictive value of the Breast Imaging Reporting and Data System. J Am Coll Surg 1999; 189:34-40.
8. Hollingsworth A B, Singletary S E, Morrow M, et al. Current comprehensive assessment and management of women at increased risk for breast cancer. Am J Surg 2004; 187:349-62.
9. Poulsom R, Hanby A M, Pignatelli M, et al. Expression of gelatinase A and TIMP-2 mRNAs in desmoplastic fibroblasts in both mammary carcinomas and basal cell carcinomas of the skin. J Clin Pathol 1993; 46:429-36.
10. Brown P D, Bloxidge R E, Anderson E, Howell A. Expression of activated gelatinase in human invasive breast carcinoma. Clin Exp Metastasis 1993; 11:183-9.
11. Zucker S, Lysik R M, DiMassimo B I, et al. Plasma assay of gelatinase B: tissue inhibitor of metalloproteinase complexes in cancer. Cancer 1995; 76:700-8.
12. Capper S J, Verheijen J, Smith L, Sully M, Visser H, Hanemaaijer R. Determination of gelatinase-A (MMP-2) activity using a novel immunocapture assay. Ann N Y Acad Sci 1999; 878:487-90.
13. Nguyen M, Watanabe H, Budson A E, Richie J P, Folkman J. Elevated levels of the angiogenic peptide basic fibroblast growth factor in urine of bladder cancer subjects. J Natl Cancer Inst 1993; 85:241-2.
14. Nguyen M, Watanabe H, Budson A E, Richie J P, Hayes D F, Folkman J. Elevated levels of an angiogenic peptide, basic fibroblast growth factor, in the urine of subjects with a wide spectrum of cancers. J Natl Cancer Inst 1994; 86:356-61.
15. Gerhards S, Jung K, Koenig F, et al. Excretion of matrix metalloproteinases 2 and 9 in urine is associated with a high stage and grade of bladder carcinoma. Urology 2001; 57:675-9.
16. Sier C F, Casetta G, Verheijen J H, et al. Enhanced urinary gelatinase activities (matrix metalloproteinases 2 and 9) are associated with early-stage bladder carcinoma: a comparison with clinically used tumor markers. Clin Cancer Res 2000; 6:2333-40.
17. Kanayama H. Matrix metalloproteinases and bladder cancer. J Med Invest 2001; 48:31-43.
18. Hanemaaijer R, Sier C F, Visser H, et al. MMP-9 activity in urine from subjects with various tumors, as measured by a novel MMP activity assay using modified urokinase as a substrate. Ann N Y Acad Sci 1999; 878:141-9.
19. Monier F, Mollier S, Guillot M, Rambeaud J J, Morel F, Zaoui P. Urinary release of 72 and 92 kDa gelatinases, TIMPs, N-GAL and conventional prognostic factors in urothelial carcinomas. Eur Urol 2002; 42:356-63.
20. Nutt J E, Durkan G C, Mellon J K, Lunec J. Matrix metalloproteinases (MMPs) in bladder cancer: the induction of MMP9 by epidermal growth factor and its detection in urine. BJU Int 2003; 91:99-104.
21. Sherief M H, Low S H, Miura M, Kudo N, Novick A C, Weimbs T. Matrix metalloproteinase activity in urine of subjects with renal cell carcinoma leads to degradation of extracellular matrix proteins: possible use as a screening assay. J Urol 2003; 169:1530-4.
22. Durkan G C, Nutt J E, Rajjayabun P H, Neal D E, Lunec J, Mellon J K. Prognostic significance of matrix metalloproteinase-1 and tissue inhibitor of metalloproteinase-1 in voided urine samples from subjects with transitional cell carcinoma of the bladder. Clin Cancer Res 2001; 7:3450-6.
23. Fang J, Shing Y, Wiederschain D, et al. Matrix metalloproteinase-2 is required for the switch to the angiogenic phenotype in a tumor model. Proc Natl Acad Sci USA 2000; 97:3884-9.
24. Roy R, Wewer U M, Zurakowski D, Pories S E, Moses M A. ADAM 12 cleaves extracellular matrix proteins and correlates with cancer status and stage. J Biol Chem 2004; 279:51323-30.
25. Iba K, Albrechtsen R, Gilpin B J, Loechel F, Wewer U M. Cysteine-rich domain of human ADAM 12 (meltrin alpha) supports tumor cell adhesion. Am J Pathol 1999; 154:1489-501.
26. Marshall L M, Hunter D J, Connolly J L, et al. Risk of breast cancer associated with atypical hyperplasia of lobular and ductal types. Cancer Epidemiol Biomarkers Prev 1997; 6:297-301.
27. Dupont W D, Page D L. Risk factors for breast cancer in women with proliferative breast disease. N Engl J Med 1985; 312:146-51.
28. Dupont W D, Parl F F, Hartmann W H, et al. Breast cancer risk associated with proliferative breast disease and atypical hyperplasia. Cancer 1993; 71:1258-65.
29. Li C I, Anderson B O, Daling J R, Moe R E. Changing incidence of lobular carcinoma in situ of the breast. Breast Cancer Res Treat 2002; 75:259-68.
30. Fisher E R, Land S R, Fisher B, Mamounas E, Gilarski L, Wolmark N. Pathologic findings from the National Surgical Adjuvant Breast and Bowel Project: twelve-year observations concerning lobular carcinoma in situ. Cancer 2004; 100:238-44.
31. Sneige N, Wang J, Baker B A, Krishnamurthy S, Middleton L P. Clinical, histopathologic, and biologic features of pleomorphic lobular (ductal-lobular) carcinoma in situ of the breast: a report of 24 cases. Mod Pathol 2002; 15:1044-50.
32. Middleton L P, Palacios D M, Bryant B R, Krebs P, Otis C N, Merino M J. Pleomorphic lobular carcinoma: morphology, immunohistochemistry, and molecular analysis. Am Surg Pathol 2000; 24:1650-6.
33. Page D L, Dupont W D, Rogers L W, Rados M S. Atypical hyperplastic lesions of the female breast. A long-term follow-up study. Cancer 1985; 55:2698-708.
34. Osborne M P B P. Atypical ductal and lobular hyperplasia and breast cancer risk. Surg Oncol Clin N Am 1993; 2:1-11.
35. Hartmann L C, Sellers T A, Frost M H, et al. Benign breast disease and the risk of breast cancer. N Engl J Med 2005; 353:229-37.
36. Renshaw A A. Improved reporting methods for atypia and atypical ductal hyperplasia in breast core needle biopsy specimens. Potential for interlaboratory comparisons. Am Clin Pathol 2001; 116:87-91.
37. Ernster V L, Ballard-Barbash R, Barlow W E, et al. Detection of ductal carcinoma in situ in women undergoing screening mammography. J Natl Cancer Inst 2002; 94:1546-54.
38. Hoogerbrugge N, Bult P, de Widt-Levert L M, et al. High prevalence of premalignant lesions in prophylactically removed breasts from women at hereditary risk for breast cancer. J Clin Oncol 2003; 21:41-5.
39. Elmore J G, Reisch L M, Barton M B, et al. Efficacy of breast cancer screening in the community according to risk level. J Natl Cancer Inst 2005; 97:1035-43.
40. Pisano E D, Gatsonis C, Hendrick E, et al. Diagnostic performance of digital versus film mammography for breast-cancer screening. N Engl J Med 2005; 353:1773-83.
41. Berry D A, Cronin K A, Plevritis S K, et al. Effect of screening and adjuvant therapy on mortality from breast cancer. N Engl J Med 2005; 353:1784-92.
42. Barlow W E, Lehman C D, Zheng Y, et al. Performance of diagnostic mammography for women with signs or symptoms of breast cancer. J Natl Cancer Inst 2002; 94:1151-9.
43. Majid A S, de Paredes E S, Doherty R D, Sharma N R, Salvador X. Missed breast carcinoma: pitfalls and pearls. Radiographics 2003; 23:881-95.
44. Brown M L, Houn F, Sickles E A, Kessler L G. Screening mammography in community practice: positive predictive value of abnormal findings and yield of follow-up diagnostic procedures. AJR Am J Roentgenol 1995; 165:1373-7.
45. Fletcher S W, Elmore J G. False-positive mammograms—can the USA learn from Europe? Lancet 2005; 365:7-8.
46. Fox S A, Murata P J, Stein J A. The impact of physician compliance on screening mammography for older women. Arch Intern Med 1991; 151:50-6.
47. Rimer B K, Ross E, Cristinzio C S, King E. Older women's participation in breast screening. J Gerontol 1992; 47 Spec No:85-91.
48. Smith R A, Cokkinides V, Eyre H J. American Cancer Society Guidelines for the Early Detection of Cancer, 2005. CA Cancer J Clin 2005; 55:31-44; quiz 55-6.
49. George S A. Barriers to breast cancer screening: an integrative review. Health Care Women Int 2000; 21:53-65.
50. Hamilton E L, Wallis M G, Barlow J, Cullen L, Wright C. Women's views of a breast screening service. Health Care Women Int 2003; 24:40-8.
51. Consedine N S, Magai C, Neugut A I. The contribution of emotional characteristics to breast cancer screening among women from six ethnic groups. Prev Med 2004; 38:64-77.
52. Fisher B, Costantino J P, Wickerham D L, et al. Tamoxifen for prevention of breast cancer: report of the National Surgical Adjuvant Breast and Bowel Project P-1 Study. Natl Cancer Inst 1998; 90:1371-88.
53. Cummings S R, Eckert S, Krueger K A, et al. The effect of raloxifene on risk of breast cancer in postmenopausal women: results from the MORE randomized trial. Multiple Outcomes of Raloxifene Evaluation. Jama 1999; 281:2189-97.
54. Vogel V G, Costantino J P, Wickerham D L, Cronin W M, Wolmark N. The study of tamoxifen and raloxifene: preliminary enrollment data from a randomized breast cancer risk reduction trial. Clin Breast Cancer 2002; 3:153-9.
55. Wickerham D L. Tamoxifen's impact as a preventive agent in clinical practice and an update on the STAR trial. Recent Results Cancer Res 2003; 163:87-95; discussion 264-6.
56. Wickerham D L. Tamoxifen versus raloxifene in the prevention of breast cancer. Eur J Cancer 2002; 38 Suppl 6:S20-1.
57. Cuzick J. Aromatase inhibitors for breast cancer prevention. J Clin Oncol 2005; 23:1636-43.
58. Goss P E. Breast cancer prevention—clinical trials strategies involving aromatase inhibitors. J Steroid Biochem Mol Biol 2003; 86:487-93.
59. Goss P E, Strasser-Weippl K. Prevention strategies with aromatase inhibitors. Clin Cancer Res 2004; 10:372 S-9S.
60. Goss P E, Strasser-Weippl K. Aromatase inhibitors for chemoprevention. Best Pract Res Clin Endocrinol Metab 2004; 18:113-30.
61. Gail M H, Brinton L A, Byar D P, et al. Projecting individualized probabilities of developing breast cancer for white females who are being examined annually. J Natl Cancer Inst 1989; 81:1879-86.
62. Claus E B, Risch N, Thompson W D. Autosomal dominant inheritance of early-onset breast cancer. Implications for risk prediction. Cancer 1994; 73:643-51.
63. Berry D A, Iversen E S, Jr., Gudbjartsson D F, et al. BRCAPRO validation, sensitivity of genetic testing of BRCA1/BRCA2, and prevalence of other breast cancer susceptibility genes. J Clin Oncol 2002; 20:2701-12.
64. Poola I, DeWitty R L, Marshalleck J J, Bhatnagar R, Abraham J, Leffall L D. Identification of MMP-1 as a putative breast cancer predictive marker by global gene expression analysis. Nat Med 2005; 11:481-3.
65. Wurtz S O, Schrohl A S, Sorensen N M, et al. Tissue inhibitor of metalloproteinases-1 in breast cancer. Endocr Relat Cancer 2005; 12:215-27.
66. Costantino J P, Gail M H, Pee D, et al. Validation studies for models projecting the risk of invasive and total breast cancer incidence. J Natl Cancer Inst 1999; 91:1541-8.
67. Gail M H, Brinton L A, Byar D P, et al. Projecting individualized probabilities of developing breast cancer for white females who are being examined annually. J Natl Cancer Inst 1989; 81:1879-86.
68 Glantz S. Primer of biostatistics, 6th ed. New York: McGraw-Hill, 2005:98-105.
69. Hosmer D W, Lemeshow, S. Applied logistic regression, 2nd ed. New York: John Wiley, 2000:47-90.
70. Breslow N E, Day, N. E. Statistical methods in cancer research. Vol. Volume 1—The analysis of case-control studies. Lyon, France: International Agency for Research on Cancer (IARC), 1980:192-246.
71. Zhou Z-H, Obuchowski, N A, McClish, D K. Statistical Methods in Diagnostic Medicine. New York: Wiley-Interscience, 2002:246-273.
72. Rosner B. Fundamentals of biostatistics, 6th ed. Belmont, Calif.: Duxbury, 2006:416-425.
73. Roy R, Wewer U M, Zurakowski D, Pories S E, Moses M A. ADAM 12 cleaves extracellular matrix proteins and correlates with cancer status and stage. J Biol Chem 2004; 279:51323-30.

1. A method for assessing breast cancer risk in a subject comprising:
(a) detecting the presence or absence of ADAM 12 in a biological sample from a subject; and
(c) detecting the presence or absence of MMP 9 in the biological sample from the subject;
wherein the presence of both ADAM 12 and MMP 9 indicates increased risk of breast cancer.

2. A method for assessing breast cancer risk in a subject comprising:
(a) measuring the level of ADAM 12 and the level of MMP 9 in multiple biological samples obtained from a subject periodically over a period time; and
(b) measuring a change in the measured level of ADAM 12 and the measured level of MMP 9 in the biological samples;
wherein an elevation in the measured level of ADAM 12 and/or measured level of MMP 9 over time indicates increased risk of breast cancer.

3. The method of claim 1, wherein the biological sample is selected from the group consisting of blood, tissue, serum, urine, stool, sputum, cerebrospinal fluid, nipple aspirates, plasma, and supernatant from cell lysate.

4-9. (canceled)

10. A method for directing medical care for a subject, comprising assessing risk for breast cancer in a subject according to the method of claim 1, wherein assessment of increased risk directs medical care comprising a secondary detection method.

11. (canceled)

12. A method for directing medical care for a subject, comprising assessing risk for breast cancer in a subject according to the method of claim 1, wherein assessment of increased risk directs medical care comprising breast cancer risk reduction.

13-17. (canceled)

18. A method for monitoring therapeutic efficacy of a breast cancer risk reduction strategy, wherein the status of ADAM 12 presence and the status of MMP 9 presence in a biological sample from a subject is used for the assessment of breast cancer risk in the subject, wherein reduction in the levels of ADAM 12 and/or the reduction in the levels of MMP 9 indicates that the breast cancer risk reduction strategy is efficacious.

19. The method of claim 18, wherein the status of ADAM 12 presence and the status of MMP 9 presence in the biological sample is measured by detecting an change in the level of ADAM 12 or the level of MMP 9 in the biological sample.

20. A method for monitoring therapeutic efficacy in a subject of a breast cancer risk reduction strategy comprising the steps of:
a. measuring the level of ADAM 12 and the level of MMP 9 in multiple biological samples obtained from a subject periodically over a period time; and
b. measuring a change in the measured level of ADAM 12 and a change in the measured level of MMP 9,
wherein a reduction in the measured level of ADAM 12 and/or the reduction in the measured level of MMP 9 over time indicates that the breast cancer risk reduction strategy is efficacious.

21. A method for assessing breast cancer risk in a subject that is at low risk for breast cancer according to the Gail 5-year risk model comprising:
(a) detecting the level of ADAM 12 in a biological sample from a subject that is a low risk for breast cancer according to the Gail 5-year risk model; and
(b) comparing the level determined in step (a) to a standard level of ADAM 12,
wherein an elevated level of ADAM 12 compared to the standard level indicates increased risk of breast cancer.

22. (canceled)

23. The method of claim 21, wherein the biological sample is selected from the group consisting of blood, tissue, serum, urine, stool, sputum, cerebrospinal fluid, nipple aspirates, and supernatant from cell lysate.

24-34. (canceled)

35. A method for monitoring therapeutic efficacy of a breast cancer risk reduction strategy, wherein the level of ADAM 12 in a biological sample from a subject that is at low risk for breast cancer according to the Gail 5-year risk model is used for the assessment of breast cancer risk in the subject, wherein reduction in the levels of ADAM 12 indicates that the breast cancer risk reduction strategy is efficacious.

36. A method to direct treatment of a subject which comprises having a subject tested for the presence of ADAM 12 and the presence of MMP 9 in a biological sample obtained from the subject, wherein a clinician reviews the results and if the biological sample is positive for the presence of ADAM 12 and the presence of MMP 9 the clinician directs the subject to appropriate medical treatment.

37. A method to direct treatment of a subject which comprises having a subject tested for the level of ADAM 12 in a biological sample a biological sample from a subject that is at low risk for breast cancer according to the Gail 5-year risk model, wherein a clinician reviews the results as compared to a standard level of ADAM 12, and if the biological sample has an elevated level of ADAM 12 as compared to the standard level the clinician directs the subject to appropriate medical treatment.

38. The method of claim 36, wherein the appropriate medical treatment is medical care comprising a secondary detection method.

39-41. (canceled)

42. A method for assessing breast cancer risk in a subject comprising:
a. measuring the level of ADAM 12 in multiple biological samples obtained from a subject periodically over a period time; and
b. measuring a change in the measured level of ADAM 12 in the biological samples;
wherein an elevation in the measured level of ADAM 12 over time indicates increased risk of breast cancer.

43. A method for assessing breast cancer risk in a subject comprising:
a. measuring the level of MMP 9 in multiple biological samples obtained from a subject periodically over a period time; and
b. measuring a change in the measured level of MMP 9 in the biological samples;
wherein an elevation in the measured level of MMP 9 over time indicates increased risk of breast cancer.

44. (canceled)

45. The method of claim 2, wherein the biological sample is selected from the group consisting of blood, tissue, serum, urine, stool, sputum, cerebrospinal fluid, nipple aspirates, plasma, and supernatant from cell lysate.

46. A method for directing medical care for a subject, comprising assessing risk for breast cancer in a subject according to the method of claim 2, wherein assessment of increased risk directs medical care comprising a secondary detection method.

47. A method for directing medical care for a subject, comprising assessing risk for breast cancer in a subject according to the method of claim 2, wherein assessment of increased risk directs medical care comprising breast cancer risk reduction.

48. The method of claim 37, wherein the appropriate medical treatment is medical care comprising a secondary detection method.
